(19) United States
(12) Patent Application Publication
(10) Pub. No.: US 2009/0221444 A1
(43) Pub. Date: Sep. 3, 2009

(54) Method for Identifying, Marking and Treating Epithelial Lung Tumour Cells and Means for Carrying Out Said Method

(75) Inventors: Jürgen Borlak, Lehrte/Ot Immensen (DE); Gabriela Salinse-Ricater, Göttingen (DE)

Correspondence Address: BUCHANAN INGERSOLL & ROONEY PC, P.O. BOX 1404, ALEXANDRIA, VA, 22313-1404, US

(73) Assignee: Anqewandlen Forschung E.V.

(21) Appl. No.: 12/092,103

(22) PCT Filed: Oct. 31, 2006
(86) PCT No.: PCT/IB2006/003959

§ 371 Date: Sep. 19, 2008

(30) Foreign Application Priority Data
Oct. 31, 2005 (DE) 102005052384.6

(51) Int. Cl. C40B 40/06; C12Q 1/70; C12Q 1/68; C12Q 1/02; C12N 5/06; C40B 30/00

(52) U.S. Cl. 506/16; 435/5; 435/6; 435/29; 435/375; 506/7

(57) ABSTRACT

The bronchial carcinoma is the most common tumour found in humans world-wide and is in most cases beyond remedy. The invention relates to a method for identifying, marking and treating epithelial lung tumour cells, specifically of an adenocarcinoma, with the aid of novel treatment targets, and to means for carrying out said method. At the basis of the invention is the discovery that the expression of genes that code for CAM and ECM molecules is modified in c-raf and/or c-myc induced adenocarcinomas of the lung. Cell-adhesion proteins and extra-cellular matrix proteins constitute the treatment targets. According to the invention, a biological or biotechnological system is brought into contact with a dissolved substance that has an affinity to at least one of the following genes: ADAM19, Mmp12, Col18a1, Col15a1, CD44, Bsg, Itgb2, Itgax, Lamc2, Lamb3, Alcam, Cldn2, Cldn3, Cldn7, Krt1-18, Krt2-8, tacstd1, tacstd2, S100a1, S100a11, their variants, parts of said genes, their mRNA or their gene products, cleavage products derived from said genes, polypeptides or peptides, said substance being bound to a suitable marker.

The invention relates to a method for identifying, marking and treating epithelial lung tumour cells, in particular from adenocarcinomas, with the help of new therapy targets, as well as a means for implementation of the method. Fields of application of the invention are diagnostic medicine, the pharmaceutical industry and molecular-biological research.

The cells of malign tumours are mis-steered, i.e. they have lost their specific properties and divide without inhibition.

In malignant tumours, a distinction is made between the carcinoma, an epithelial tumour coming from the covering tissue, with about 90% of all tumours being of epithelial origin, and the sarcoma, a mesenchymal tumour coming from the connective tissue, which makes up about 10% of all tumours.

In general, lung cancer is understood to mean a degeneration of the tissue in various areas of the lung. This includes not only a bronchial carcinoma (cancer of the actual lung tissue), but also very rare cancer diseases such as the mesotheliom (cancer of the pleura).

Bronchial carcinomas occur most frequently between the 65^{th} and 70^{th} year and are the most frequent form of cancer in men in Germany. Each year, more than 42,000 people contract lung cancer, around five percent of those affected being younger than 40. In about 90 percent of the cases, smoking is the cause for the origination of this disease. World-wide, bronchial carcinomas are the most frequent tumour in humans. Lung cancer is a very severe disease which is beyond remedy in most cases up to now.

Adenocarcinomas are the most frequent malignant disease in men and women. As with other malignant tumours, the survival period of the patients is influenced by a number of factors, inter alia by the formation of metastases. In order to determine prognostic factors and to prevent metastasising of tumours, new approaches for an improved therapy are being developed world-wide. Cell adhesion and extra-cellular matrix molecules as well as metalloprotinases and their inhibitors are distinctly interesting targets for tumour therapy. The expression of adhesion molecules to the cell surface is subject to a series of changes. Cell-cell and extra-cellular matrix interaction determined by adhesion molecules have a decisive influence on the formation of tumours and metastasising of malignant new formations.

The invention was based on the task of stating a simple, sensitive and gentle method for identifying, marking and treating epithelial lung cancer cells and of providing and using a means of their identification, marking and treatment.

This task is solved by a method pursuant to the features of claim 1 and means pursuant to claim 26 for the method according to the invention and a test kit pursuant to claim 28. The sub-claims are further formations of the invention.

The focal point of the invention is the knowledge that the expression of genes coding for CAN and ECM molecules is amended in adenocarcinomas of the lung induced in c-raf and/or c-myc.

The invention is based on the discovery of new therapy targets. This is a question of cell adhesion and extra-cellular matrix proteins which are suited for an antibody therapy of all carcinomas of the lung, e.g. of adenocarcinomas. Unlike conventional polychemical therapy with minimum success rates (as about 80% of the patients die within the first year after the diagnosis of the lung tumour diseases), the identification of new therapy targets, which succeeded totally surprisingly, has made a selective and gentle therapy and diagnosis possible for the first time.

In the studies which led to the invention, adenocarcinomas of the lung were examined in transgenic mice. It was seen that basigin, an inductor of the extra-cellular matrix metalloproteinase, was over-expressed, whereas the expression of a basigin inhibitor and tumour suppressor, caveolin, is repressed. In this way, the induction of ADAM-19 was proven by expression of the coding gene and the translated protein. It is a question of a new kind of “disintegrin” matrix metalloproteinase. As a result of repression of TIMP3, an inhibitor of ADAM proteins, the tumour procures considerable advantages. For example, ADAM-19 organises the decomposition of, inter alia, collagen 17alpha1 and thus supports tumour integration. This process is prevented by TIMP3, which is repressed in adenocarcinomas of the lung. In addition, the embodiments of the invention show that other, further cell adhesion molecules are strongly repressed, for example cadherin 2, 5 and 13; procadherin alpha 4; procollagen 17a1 and 13a1; laminins 2, 4 and 1; integrin alpha 8; ICMA2; vinculin; vitronectin and tetrapsin. On the other hand, Ep-CAM was induced. The selective expression of cell adhesion molecules leads to amended signal transmissions, which support the cell migration and proliferation of tumour cells. Further, it was seen in the course of the work on the invention by immunohistochemical examinations on human tumour tissues of the lung, that the therapy targets being described here for the first time are also strongly expressed or regulated in a tumour-specific way (both in non-small-cell and also in small-cell tumours with a varying degree of differentiation) in carcinomas of the human lung. This is why the knowledge gained from the animal model was transferred directly to humans. Thus, there are already first clinical studies for the Ep-CAM adhesion molecule for the treatment of mammary and colon carcinomas. The invention is equally suited to therapy with inhibitory antibodies as well as bifunctional antibody (recruiting of cytotoxic CD4+ or CD8+ cells). But also antibodies with cytotoxic conjugates as well as active ingredient molecules can be developed purposefully for the new kind of therapy targets pursuant to the invention and therefore be used therapeutically.

Some of the principles of such a possible tumour model have already been described.

Therefore, the basis of the invention is the regulation of adhesion molecules and MMPs as well as their inhibitors in c-raf and/or c-myc induced lung adenocarcinomas, which was discovered completely surprisingly.

For this, c-raf (or c-myc) was used as an essential integral part of the mitogen and stress-induced signal transduction paths. Its directed over-expression in the alveolar epithelium caused adenocarcinomas. Tumour growth was put into relation with amended expression of molecules of the extra-cellular matrix (ECM) and the cell adhesion (CEM). In the course of the invention, the induction of basigin, an inducer for extra-cellular matrix metalloproteinases, was observed, whereas its inhibitor and tumour suppressor Caveolin-1 was repressed. In addition, the expression of ADAM-19, a new kind of disintegrin metalloproteinase, was increased, whereas that of TIMP-3, an inhibitor of ADAM proteins, was suppressed.

This facilitates the decomposition of collagen17a1 and consequently of cell migration.

Further, a drastic repression of numerous CAM molecules (CAMs) including Cadherin 2, 5, 13, Procadherin alpha 4, procollagen type XIII and XVII, laminins (2.4.1), integrin alpha 8, ICAM 2, vinculin, vitronectin and tetrapsins was observed, although the expression of Ep-CAM was distinctly increased. Above all, selective expression of CAMs changes the cellular signal transmission and the newly designed CAM and ECM molecules make new kinds of mechanism-based therapies of lung cancer possible.

In this way, a method with the help of which sensitive epithelial lung cancer cells of adenocarcinomas can be simply identified, marked or treated in a biological or biotechnological system was made possible for the first time. The biological system essentially means organisms, cell tissue, cells, integral parts of cells, DNA, RNA, cDNA, mRNA, cRNA, proteins and/or peptides and well as structures derived from them, the biotechnological system being all possible examination arrangements, with the help of which properties of epithelial tumour cells of lung adenocarcinomas or structures derived from them can be proven.

For this, the biological or biotechnological system is put into contact with at least one substance, which has affinity to at least one of the following components: to the genes ADAM19, Mmp12, Col18a1, Col15a1, CD44, Bsg, Itgb2, Itgax, Lamc2, Lamb3, Alcam, Cidn2, Cidn3, Cidn7, Krt1-18, Krt2-8, tacstd1, tacstd2, S100a1, S100a11 and/or their variants and/or parts thereof and/or their mRNA and/or their gene products and/or cleavage products, polypeptides or peptides derived therefrom. The focal point of the invention in this connection is that the substance is connected with a marker, which possesses a certain property.

The method is particularly suited if the biological or biotechnological system entails or contains an organism, cell tissue, cells, cell components, DNA, RNA, cDNA, mRNA, cRNA, proteins or peptides or structures derived therefrom, as they are particularly well suited for identifying, marking or treating epithelial tumour cells from lung adenocarcinomas with the substance marked according to the invention, in particular if they entail lung tumour cells and/or oligonucleotide libraries.

As at least partly soluble substances, oligonucleotides, proteins, peptides or structures derived therefrom are particularly suited. They have the benefit that they can specifically identify two or three-dimensional target structures on a molecular level. Over and above this, they have the beneficial property that identification takes place as a rule in aqueous, physiologically buffered solutions and leads to a specific association/binding with the target structure.

In this context, monoclonal and/or polyclonal antibodies or fragments of antibodies are particularly suitable, as they are formed as robust, highly specific structures which principally can be produced with affinity against all possible molecular target structures.

In a beneficial further formation of the invention, human and/or bi-specific antibodies or human and/or bi-specific fragments of antibodies are used, as they at least can reduce the immune response of the human immune systems in contact with the substance in accordance with the invention or can connect macrophages or other components of the immune system with a target structure, in particular epithelial lung tumour cells from adenocarcinomas as a result of the bi-specific binding.

In particular, monoclonal antibodies and/or fragments of antibodies are suited in this context as they permit a particularly good selectivity, sensitivity and reproducibility of the method according to the invention.

It is particularly favourable for the implementation of the invention if the marker according to the invention has been formed as an element, isotope, molecule and/or ion or has been composed of the latter, in particular has been formed as a dye, contrast agent, chemo-therapeutic, radionuclide, toxin, lipid, carbohydrate, biotin, peptide, protein, microparticle, vesicle, polymer, hydro-gel, cell organelle, virus and/or whole cell or entails the latter, by which the marked substance can be adapted to the various requirements. If, for example, biotin is used as a marker, the quantity of the necessary pharmacon added in a second/subsequent step can be reduced in an examination and/or a therapy of epithelial lung tumour cells, in particular from adenocarcinomas, if the pharmacon is bound to streptavidin.

It is also particularly suitable if the marker has been formed as a preferably dye- and/or enzyme-marked secondary antibodies and/or protein A and/or protein G or structure derived therefrom or contains the latter.

For example, it is also possible to use hyaluronan or derived structures as markers, as it can favourably be absorbed by target structures/receptors of the affine substance at the same time.

The connection between the marker and the substance is beneficially organised chemically, electrostatically and/or via hydrophobic interactions, as they manifest sufficient binding stability for the use of the marked substance for identifying, marking and treating epithelial lung tumour cells, in particular from adenocarcinomas, preferably if the connection is covalent. In this context, the electrostatic and/or hydrophobic interactions emanating from or formed between amino acid residues or their side chains, preferably proteins, or nitrogen bases of nucleotides, are particularly suited in this context.

To increase the sensitivity of the method according to the invention, simultaneous use of a number of substances is particularly suitable, in particular if they entail three substances with affinity to Bsg and Cidn2 and Tntsf9 or their mRNA sequences or their gene products.

For a particularly specific identification, substances which bind to the target structure with an affinity above the association constant Ka=1,000 M−1 are particularly suited.

For various uses of the method pursuant to the invention, it is beneficial to use one of the following methods —PCR, in vitro translation, RT-PCR, gel electrophoresis, Western Blot, Northern Blot, Southern Blot, ELISA, FACS measurement, chromatographic separation, UV microscopy, immunohistochemistry, screening of solid-phase bound molecules or tissues and/or biosensory examination—in which context a particularly simple implementation of the method according to the invention is made possible by reproduction, separation, immobilisation and/or detection of the example sample and of parts thereof, in particular also when a statistical analysis is carried out.

According to another further formation of the invention, molecules, cells and/or tissues which are immobilised on a planar surface, preferably locally addressed, are used for the implementation of the method in accordance with the invention. The immobilisation has the advantage that it enables particularly easy identification, marking of lung tumour cells, as surfaces can be screened particularly simply, in particular if it has been formed as a membrane, e.g. from nitrocellulose or PVDF, as cavity of a microtitre/ELISA plate or a cell culture vessel or as a glass or plastic chip.

Beneficially, substances which have affinity to at least one of the genes ADAM19, Mmp12, Col18a1, Col15a1, CD44, Bsg, Itgb2, Itgax, Lamc2, Lamb3, Alcam, Cidn2, Cidn3, Cidn7, Krt1-18, Krt2-8, tacstd1, tacstd2, S100a1, S100a11 and/or their variants and/or parts thereof and/or their mRNA and/or their gene products and/or cleavage products, polypeptides or peptides derived therefrom (hereinafter called “target structure”) are used as solid-phase-bound molecules for this purpose, as they permit a particularly simple identification or examination of epithelial lung tumour cells on the surface if at least one of these substances interacts with their target structure, in particular binding to the latter. In particular, immobilised molecules provided as an oligonucleotide, antibody or antibody fragment are particular suited for this.

The detection of target structures, e.g. of receptors on an immobilised tissue cut or on cells of a cell culture, can be done with any marked molecules subsequently put into solution onto the surface, advantageously with one of the marked substances according to the invention, for example a (fluorescence) marked antibody.

If an RT-PCR is done, corresponding oligonucleotide probes and primers are beneficially applicable.

If immobilised molecule libraries, for example DNA or antibody libraries interacting with the target structures from a solution, for example with a cDNA library, a PCR product library or with epithelial lung cancer cells, in particular from adenocarcinomas, are used for the implementation of the method according to the invention, the target structures bound to the molecule libraries can be particularly well detected as a result of the use of (dye-) marked probes subsequently added, preferably oligonucleotides or structures derived from them. If unmarked primary antibodies are used for detection, they can favourably be detected with marked secondary antibodies. But also the use of other proteins, for example enzymes, or streptavidin or parts thereof is suited.

For the diagnostic detection of epithelial lung cancer cells, in particular from adenocarcinomas, in in-vitro and in-vivo diagnostics with the help of the method according to the invention, use of optical apparatus, e.g. UV microscopes, scanners or ELISA readers, photometers or of scintigraphic apparatus, e.g. X-Ray appliances, is preferably suitable.

A further aspect of the invention relates to a means of identifying, marking and treating lung tumour cells, in particular epithelial lung cancer cells, in particular from adenocarcinomas, which contains a substance with an affinity pursuant to claim 1 and, if applicable claim 14, and pursuant to the features according to one of claims 4-6 and where at least one of the substances is connected with a marker pursuant to one of the claims 7-9, if applicable via a connection pursuant to claims 11-12, and said possibly marked substance is at least partly soluble, in particular in aqueous or physiological solutions.

According to a further aspect of the invention, the corresponding means is used for the production of a preparation for sensitive and gentle identification, marking and/or treating of lung tumour cells, in particular from epithelial lung cancer cells from adenocarcinomas, which is particularly simple to implement.

Another aspect of the invention relates to a test kit for the implementation of the methods according to the invention for diagnostic identification, marking and/or test treatment of epithelial lung tumour cells, preferably from adenocarcinomas, in which context the test kit contains at least one means according to the invention pursuant claims 26-27, in particular if the affine substances of the means are immobilised to a surface, preferably locally addressed.

Some of the particularly favourable features and further beneficial properties of the invention become apparent from the following description and closer explanation.

The raf-kinases are proto-oncogens, which process signal cascades at the entering point of the MAPK/ERK (mitogen-activated proteinkinase/extra-cellular signal-regulated kinase) and signalises to the molecules that cell surface proteins and raf-proteins are to be combined with the cell core. In particular, raf-1 is activated by a large number of growth factors, hormones and other external signals. After activation, raf-1 acts on MEK and phosphorolyses it and bounds it to ERK, which is also activated by phosphorolysation [1,2]. The ras/raf/MEK/ERC cascade is regarded as a linear sequence, with ERK as the functional endpoint [3,4]. Activated ERK can be translocated to the core and thus forms a direct mediation between an extra-cellular signal, an internal sequence of signals and the core response [4]. Over-expression of raf is associated with malignant tumours [5,6] and with the tumour growth they require an amended cell-cell contact and cell-matrix adhesion, in order to make expansion, invasion and tumour preparation possible.

The examination of the adhesion molecules can thus make insights into the basic principles of tumour genesis possible and facilitates the identification of targets for new kinds of therapy forms.

In general, adhesion molecules are sub-divided into cadherins, into integrins, immunoglobulin-like receptors, CD44 and in catenins [7]. These molecules function in a complex and coordinated way, in order to keep cells at one place or alternately to support cell movement.

Further, adhesion molecules act on bi-directional signal paths, which are needed for many cellular functions, including transcription, cytoskeletal organisation and proliferation [8,9].

In particular, integrins are a main group of adhesion receptors of the cell surfaces, which support a mechanical transmembrane connection to the cytoskeleton and which activate a number of signal cascades.

In this way, integrins influence cell behaviour, including form, motility, differentiation, proliferation and survival [9].

Over and above this, matrix metalloproteinases (MMPs) represent a family of secreted, zinc-dependent endopeptidases, which are jointly capable of degradation of the integral parts of the extra-cellular matrix (ECM), and there are convincing indications of the fact that MMPs play an important role in the various steps of tumour growth.

In fact, MMPs lyse partly decomposed fine-fibred collagens and elastine and show a high affinity to collagens which are important integral parts of the basic epithelial membranes and which are lysed during tumour growth. In addition, tumours frequently show an increase of expression of MMPs and a decrease of their inhibitors, e.g. of TIMPs, which lead to an increase of proteolytic activity [10,12]. In addition, cell surface proteoglycanes (PGs) are important for the modulation of cell adhesion and motility.

Many adhesion-supporting components within the structure proteins containing ECM (laminins and collagens) as well as the cell surface adhesion receptors (CAMs) support cell adhesion via interactions with PGs.

The fact that PG connection points exist in the direct vicinity to integrin, to cell-binding domains within the ECM molecules and on other cell surface adhesion molecules, suggests that cellular identification of the ECM is involved in the formation of receptor complexes and that they modulate differing, but overlapping signal transduction paths within the cells [13,14,15].

As one result of the c-raf over-expression, lung adenocarcinomas were formed by transgenic mice. Within the work relevant for the invention, gene expression profiles were determined in histologically defined adenocarcinomas and the transcription compared with normal lung tissues of non-transgenic mice. Corresponding work was also carried out with c-myc (over-expression).

428 transcripts in lung adenocarcinomas were established as being differently expressed (p figure, t-test: <0.05). 72 genes coded for components of the ECM, of which 20 were over-expressed (table 1), whereas 52 transcripts were repressed (table 2a).

Table 1 shows the transcripts which were identified in adenocarcinomas as being over-expressed.

Remarkably, the selective expression of claudin 2 was outstanding in lung adenocarcinomas. Its expression is not found in normal lung tissue. Further, claudin 3 and 7 were over-expressed in the tumour tissue. Therefore, the claudin expression in lung adenocarcinomas, induced by c-raf 1, had been changed. Remarkably, the alveolar epithelial cells of rats express claudin 3, 7 and 5, in which context claudin 5 is strongly expressed through the entire alveolus. Unlike this, the expression of claudin 1 and 2 is minimal or completely absent [16,17]. claudins are components of the so-called tight junctions (TJs) and are epithelial barriers functioning by para-cellular permeability. Previous studies on claudin distribution suggest various barrier functions in various epithelial cells and endothelial tissue and also that claudins are selectively expressed for changes of the permeability. Changes of the communication within the TJs is an important molecular mechanism in the progression of lung tumours. This is supported by newer studies, which suggest that claudin 1 and 2 forward the activation during cancer invasion across all membrane type MMPs [1,8].

Further molecules which can be used for the classification of lung tumours were established, and they include keratin 1-18, a marker for lung adenocarcinomas which has already been described.

Calcium-binding proteins (S100A1 and S110A11) were likewise induced. The S100 family is one of the largest sub-families of calcium-binding proteins and is characterised by highly preserved helix-loop-helix regions, known as “EF-hand” motives. S100 are included in the tumour genesis and provide calcium signals during growth, differentiation and motility of the cells [19].

In addition, CD44 was induced as a result of the c-raf over-expression. This receptor is a surface glycoprotein which is involved in cell-cell and cell-matrix interactions.

Over-expression of CD44 was connected with the growth and the proliferation of a series of differing cell types in the tumour development. CD44 acts as a reproducible receptor for hyaluronan, which is internalised by endocytosis and metabolised. CD44 can anchor epithelial cells to hyaluronan in basic membranes for obtaining of polar orientation [20]. This receptor can also function by leukocyte adhesion and rotation on endothelial cells, which point them to peripheral lymphoid organs and points of inflammation and also to leukocyte aggregation. Signalisation by CD44 induced release of cytokine and T-cell activation [21].

### Over-Expression of Ep-Cam in Lung Adenocarcinomas

The discovery of over-expression of tumour-associated calcium signal transductor (tacstd) or of Ep-CAM was an important result, both with a view to their properties as adhesion molecules and function as a therapy target.

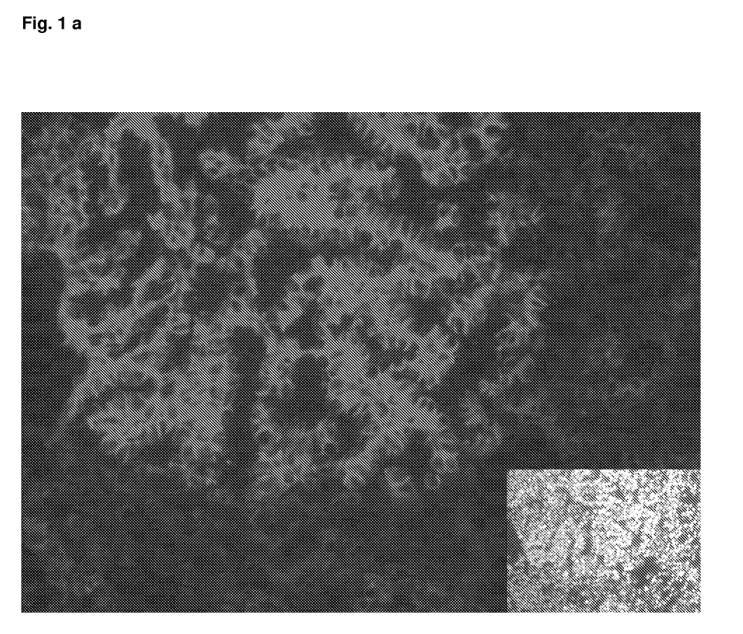

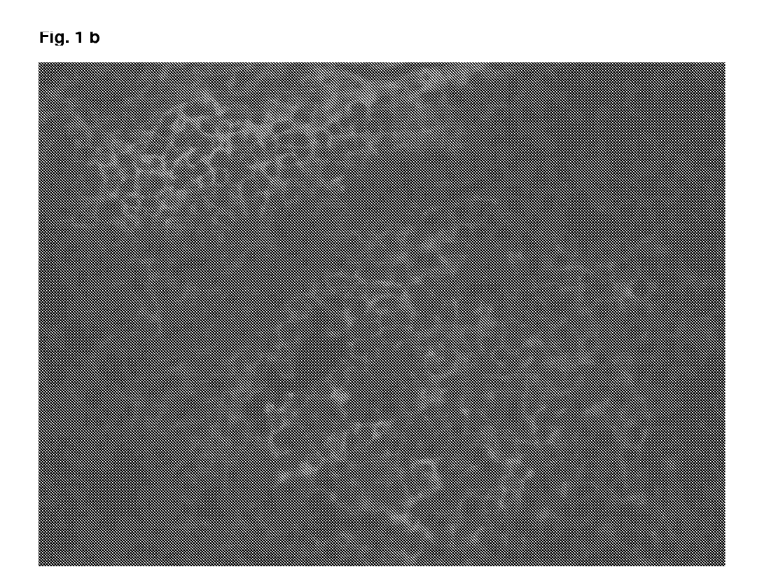

As shown in FIG. 1, the Ep-CAM immunofluorescence marking above the background level is limited by a channel tree. Membrane dyeing is seen in the respiratory epithelial lines and terminal bronchiae and bronchioles (FIG. 1a). Further, Ep-CAM dyeing emphasises the tumour centres (FIG. 1 b). The response is homogeneous and membrane-bound. The hyperplastic alveolar epithelium in the centres is dyed equally positively.

The high expression of Ep-CAM in both colon and also other epithelial carcinomas is important, and it can be assumed that this a new type of adhesion molecule which is structurally not similar to the members of the four main families of the immunoglobulin super-family (cadherins, integrins, selectins and CAMs).

Recently published indications suggest that the extra-cellular domain of Ep-CAM comprises a region rich in cystein, which contains two Type II EGF (epidermal growth factor)-like repetitions which are followed by a region low in cystein [22].

Increased Ep-CAM expression is connected with proliferation, reduced cadherin-induced adhesion and a less differentiated phenotype, which results in the suggestion that Ep-CAM regulates the strength of the intra-cellular adhesion and generates epithelial cells with flexible intermediate connections, which are necessary for epithelial morphogenesis and maintenance of tissue.

The data recently found with regard to the organisation of Ep-CAM induced adhesions indicate that it is more similar to a typical adhesion molecule which is connected to actin-microfilaments [22].

### Redistributed Adhesion Molecules in Lung Adenocarcinomas

Integrins are important ECM receptors and are also involved in cell-cell interactions. There are a number of reports indicating the critical role of integrins in tumour origination, invasion and metastasising in lung adenocarcinomas. The expression of genes which code for molecules for cell adhesion and cell-cell contacts was induced, e.g. by integrins (Itgb2 and Itgax), procollagen XV (Col15a1) and laminins (Lamb3 and Lamc2). More than 30 transcripts coding for cell adhesion molecules, containing vitronectin (Vtn), vinculin (Vcl) and intra-cellular adhesion molecule 2 (Icam2), integrin-induced cell adhesion and ECM receptors are repressed in lung adenocarcinomas.

In particular, integrin alpha 8 (Itga8) was significantly repressed. The work according to the invention shows that integrin alpha 8 plays an important role for the maintenance of tissue integrity during an injury or the regulation of mesangial cell phenotype. Integrin alpha 8 also supports adhesion and prevents the migration and proliferation of mesangial cells [23].

Further, a number of molecules interacting with integrins were selectively changed. In fact, tetrapsins, e.g. TM4SF 1, 2, 6, 12 and 13, were to be found amongst the most dramatically reduced gene transcripts in the lung adenocarcinomas induced by c-raf over-expression. The same was also established for c-myc (over-expression).

These cell surface proteins pass through the membrane four times and are found on many differing cell types of many organisms. They show numerous properties in cell adhesion, motility and proliferation and are involved in metastasising or viral infections [24].

Members of the TM4SF family also interact with adhesion receptors of the integrin family and regulate integrin-dependent cell migration [25]. The work in accordance with the invention shows, for example, the loss of gene expression of TM4SF of lung adenocarcinomas in comparison with normal lung tissue. Therefore, knowledge of the properties which keep tetrapsins together and how a loss of a tetrapsin can influence the functions of its partner molecules is of very great importance.

The repression of many cadherins (procadherin alpha4, cadherin 2, 5 and 13) was a further outstanding result of c-raf induced or c-myc induced lung adenocarcinomas. Reduced expression of E-cadherin is regarded as one of the main molecular incidents involved in the dysfunction of the cell-cell adhesion system and which make cancer invasion and metastasising possible. Research work on E-cadherin has given an insight into embryogenesis and oncogenesis. The control of epithelium-mesenchymal conversion is stated as being one of the most important functions of E-cadherin in development [26].

### Selective Regulation of Basigin, ADAM-19 and TIMP-3 in Lung Carcinomas

Tumour progression is a multi-phased process, in which cellular motility is associated with controlled proteolysis and which contains interactions between tumour cells and the ECM. Cells transformed during the tumour growth migrate from the primary tumour to cross-structured barriers, comprising basic membranes and surrounding stromal collagenic ECM. Decomposition of stromal ECM is assumed as being essential in tumour-induced angiogenesis. High-degree expression of MMPs in tumour tissues is brought into connection with tumour invasion and progression.

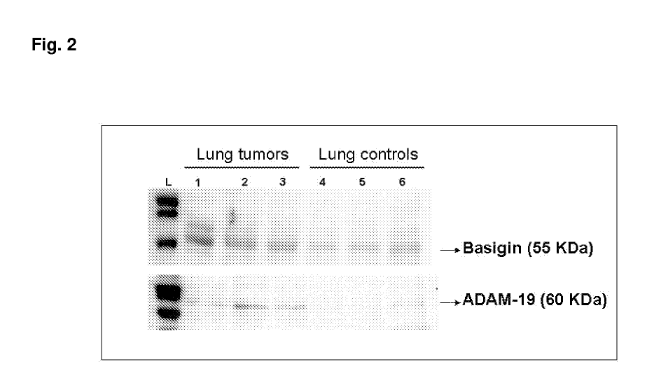

Within the work according to the invention, basigin or EMMPRIN (extra-cellular matrix metalloproteinase inducer) was highly regulated both on the gene and also on the protein level (FIG. 2). Basigin is a cell surface trans-membrane glycoprotein, which is expressed in great proliferation on many differing types of cells and appears to be particularly increased in invasive tumour cells [27-28]. Increased basigin expression correlates well with the tumour progression of glioma cells [29,30], hepatoma cells [31], flaky cell carcinomas [32] and melanomas [33,34]. Basigin stimulates the production of MMPs and transports the hyaluronan production in breast cancer carcinomas [35]. Basigin is at least partly regulated by caveolin 1 (cav1), a well established tumour suppressor, and was found repressed in lung adenocarcinomas of c-raf transgenic mice in the work done for the invention. Caveolin 1 associates with basigin during biosynthesis in the Golgl complex and escorts basigin to the cell surface, by which self-aggregation of basigin and induction of MMPs in inhibited [36]. The difference in the gene expression between basigin and caveolin 1 indicates that basigin favours the production of MMPs in c-raf-1 induced lung adenocarcinomas. The same was also observed for c-myc.

Further, ADAM-19 was established as over-expressed within the work which led to the invention. ADAM proteins are a family of Type I transmembrane glycoproteins, which contain disintegrin and metalloprotease domains (A Disintegrin And Metalloprotease) and which fulfil functions in the fertilisation, heart development neurogenesis and so-called protein ectodomain shedding [37].

ADAM proteins are known to be expressed in keratinocytes and are actively involved in keratinocyte motility. Collagens therefore appear as a new kind of substrate calls for ADAM [38,39].

The extracellular matrix metalloproteinase inducer basigin and ADAM-19 were both examined with the help of western blots, and both gene products were increased in lung adenocarcinomas induced by c-raf (FIG. 2). Analogous results were also obtained for c-myc.

Collagenic transmembrane proteins are further expressed and are involved in cell adhesion and epithelial/mesenchymal interactions during morphogenesis. Collagens XIII and XVII were established as being repressed in accordance with the invention. They are components of morphologically differing cell adhesion structures, for example of focal adhesion or, for example, hemidesmosomes.

Collagen XVII, a Type II transmembrane protein and epithelial adhesion molecule, can be discarded proteolytically from the cell surface in order to general soluble collagen.

Discarding by so-called shedding is increased, for example, by phorbol ester and inhibited by metalloprotease inhibitors, for example by hydroxamates or TIMP-3, but not by inhibitors of other protease classes or by TIMP-2 [40]. Functionally, the distribution of the ectodomains of collagen XVII (Coli 7a1) is connected with a change in the collagen motility in vitro [41].

In addition, the identification of a reduced expression of tissue inhibitors of metalloproteinase-23 (TIMP-3) was an important result during the work on the invention. More recent studies have described TIMP-3 as an endogenous inhibitor of ADAM-17, and only very recently there was a report of an inverse expression pattern of ADAMs and TIMP-3 in pathogenesis of prostate cancer [42].

Remarkably, deletions of TIMP-3 interrupt the outstanding TIMP/MMP balance which is required for proper focal ECM proteolysis and leads to the correct morphogenesis of the bronchiole branching in the development of mice lungs [43]. In addition, TIMP-3 merely shares a 25% amino acid homology with TIMP-1 and TIMP-2.

As has been shown, repression of TIMP-3 supports the tumour cell invasivity in vitro and the induction of tumour growth in vivo [44]. Although it has been shown that TIMP-3 inhibits the endothelial cell migration and tubule formation in vitro, the effect of TIMP3 over-expression on tumour-induced angiogenesis in vivo has yet to be examined.

To sum up, ADAM-19 is a sheddase candidate for Col17a1 in the lung adenocarcinoma and TIMP-3 inhibits proteolysis of Col17a1. The biological effects of collagen XVII discarding have yet to be completely clarified. A first forecast is that the separation of the ectodomains favours the release of keratinocytes during morphogenesis, differentiation and regeneration and also participates in the adhesion of basal keratinocytes to the extra-cellular matrix.

Over and above this, a number of molecules associated with cell-induced immune and inflammation processes in lung adenocarcinomas were expressed in deviation (Table 2b). For example, an over-expression of Ifl30, Ccr5, Ccl6, Ccl21b, colony stimulating factor receptor (Csf2ra, Csfrb1 and Csf2rb2) and of tnsf9 was observed. The increased production of IFH-gamma induced protein 30 (Ifl30) stimulates the synthesis of MHC Class II molecules, amongst them H2-Dma, H2-DMb1, H2-Abl and H2-Aa, selectively supporting the transmigration of Th1, but not of Th2, into endothelial cells. This establishment within the framework of the invention is extremely interesting for Th1-type selective transmigration along the epithelial cells via CCR4 [45].

A regulation of the TNF (ligand) superfamily member 9 (TNFsf9) was also observed, a cytokine which belongs to the tumour necrosis factor (TNF) ligand family. Tnfsf9 is a ligand for TNFRSF9/4-1BB, which is a co-stimulatory molecule in T-lymphocytes. This cytokine and its receptor are involved in an antigen-presenting process and the generation of cytotoxic T-cells.

Tnfsf9 was induced in c-raf lung carcinomas and was also found in increased quantities in various carcinoma cell lines. Analogous results were also observed for c—myc.

Tnfsf9 is involved in T-cell/tumour cell interaction [46] and was not found in normal mouse lung tissue.

Within the work done for the invention, the results obtained with the help of the gene chip microarray were confirmed by the use of RT-PCR. The primer sequences used can be seen from the embodiments of the invention. Table 3 shows the results for 12 of the genes which are described in the change of the extra-cellular matrix and cell-induced immunity in lung adenocarcinomas, whereas FIGS. 1 and 2 portray the expression of selected proteins.

### Discussion

Within the work which led to the invention, the re-formation of the ECM and cell adhesion molecules into lung adenocarcinomas was examined with the help of microarray, qRT-PCR, immunohistochemistry and Western Blots. As a part of this study, the expression of integrins, which form a large family of heterodimeric cell surface receptors and are involved in the cell/extra-cellular matrix and cell/cell adhesion communication, was analysed. Of the 24 differing integrin receptors which are currently known (each with a specific ligand binding and signal properties), five act as collagen receptors. Itgax is one of the integrins which can bind to collagens. In lung adenocarcinomas, the repression of transmembrane collagens (colXIIIa1 and colXVIIa1) and the over-expression of collagens which are associated with membrane fixing, such as colXVIIIa1 and colXVa1, are found. Collagens XV and XVII entail the multiplexin subfamily and non-fibrillary collagens. The over-expression of collagens XV and XVII therefore presumably contributes to cell migration of lung adenocarcinomas. These differences in the migration mechanisms can be very helpful in explaining some of the cell-specific effects of integrins on cell migration.

As shown by the embodiments, tumour growth and progression is supported by the re-structuring of the surrounding ECM. Decomposition of the transmembrane molecules colXVIIa1 is presumably catalysed by ADAM-19 in lung adenocarcinomas. The decomposition of colXVIIa1 is controlled by TIMP-3, a specific inhibitor of ADAM17 and also of other members of this family. TIMP-3 is therefore in connection with the amended ECM and is involved in the endothelial cell migration and angiogenesis. The interactions of cells with the ECM is decisive for the normal development and function of an organism. Therefore, the regulation of the composition and completeness of the ECM structure is of decisive importance. Enzymes which are involved in the restructuring play an essential role in the control of signals which are achieved by matrix molecules which control cell proliferation, differentiation and cell death.

Over and above this, tight junctions produce the connection between epithelial and endothelial cells, which include the various tissues in the body. They regulate the passage of molecules through these natural barriers. Epithelial transport takes place through trans-cellular and para-cellular routes, some of which are regulated by claudins.

In particular, there are at least 20 genes which code for claudin proteins with differing, tissue-dependent expression and barrier function.

Within the work on the invention, an over-expression of claudin 2, 3 and 7 was seen in lung adenocarcinomas. For example, claudin 2 is selectively expressed in lung adenocarcinomas, whereas claudin 5 expression is repressed in lung tumours. Generally speaking, the removal or addition of claudins influences the permeability properties of the tight junctions, as they form para-cellular pores or channels, which control the selective ion permeability (Anderson 2001 and Tsutika et al. 2001). Taking into account that the nature of the channel-forming claudin determines the specificity and selectivity for ions, the displacement of expression of claudin 5 to claudin 2 is now shown for the first time, a highly remarkable change in the pattern for claudins, which are responsible for the changes in the para-cellular routes and maintenance of cell polarity in lung adenocarcinomas.

The starting point for the invention is therefore the restructuring of ECM and CAM as an incident of the c-raf (or c-myc) over-expression in the alveolar epithelium, which was established completely surprisingly.

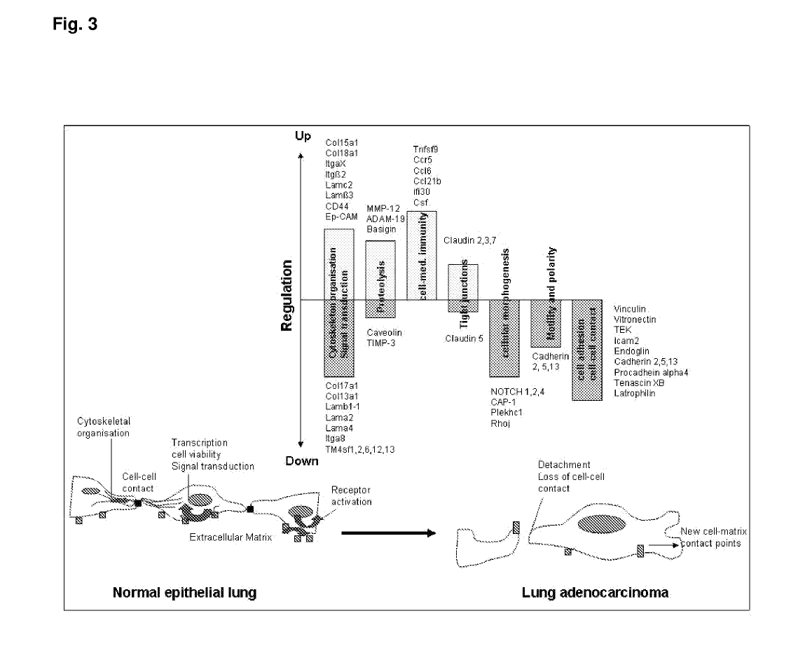

The invention is based on the knowledge, obtained for the first time, that the expression of ECM and CAM molecules is associated with expansive tumour growth and tumour invasion (FIG. 3). Amongst them, upward regulation of Ep-CAM is particularly over-clear, which are thus available for the first time as target structures in lung adenocarcinomas, for example for antibody-based therapy forms.

### EFFECTS OF THE INVENTION

The invention based on the establishment of new therapy targets now makes a selective, efficient and gentle diagnosis and therapy of lung tumour cells possible for the first time. Over and above this, the invention enables a better effectivity of precautionary examinations for early identification of changes to the lung tissue, which can easily be applied in routine diagnostics. The therapy targets are cell adhesion and extra-cellular matrix proteins, which are suitable for a therapy, in particular antibody therapy, of all carcinomas of the lung, e.g. of adenocarcinomas.

Unlike conventional polychemical therapy with minimum success rates (as about 80% of the patients die within the first year after the diagnosis of the lung tumour diseases), the completely surprising identification of new therapy target structures enables selective and gentle therapy and diagnostics for the first time. Alongside the new therapy targets, the use of substances with affinity to the described repressed genes (see Table 2a) or gene products or structures derived therefrom is in particular sensible, as they make a further (control) possibility for the identifying, marking and treating of tumour cells feasible.

Equally, the invention forms a basis for therapy with inhibitory antibodies as well as bifunctional antibodies (recruiting of cytotoxic CD4+ or CD8+ cells). But also antibodies with cytotoxic conjugates as well as active ingredient molecules can be developed purposefully for the new kind of therapy targets pursuant to the invention and therefore be used therapeutically.

Further details and methods for the implementation of the invention become more clearly visible from the following embodiments.

Extraction of total RNA. For the RNA preparations, normal lung tissue (n=6) and tumoral lung tissue of raf-1-BxB-23 transgenic mice (n=8) were used. The RNA extractions and purifications were done with an RNeasy midi kit (Qiagen, Cat. no.:751414) according to the manufacturer's instructions.

cDNA synthesis. First-strain cDNA was synthesised from 10 μg of total RNA with an oligo(dT)24 primer (PROLIGO Primers and Probes; SuperScript II Rnase H-Reverse transcriptase, 5× first-strain buffer and 0.1 M DTT (Invitrogen; 18064-014 or 18064-071), dNTP Mix, 10 nM (Invitrogen; 18427-013). The second-strain synthesis was carried out in 20 μl of the first-strain reaction mixture for 1 h at 42° C. making use of 5× second-strain buffer (Invitrogen; 10812-014), DNA ligase E. coli, 10 U/μl (Invitrogen; 18052019), DNA polymerase I E. coli, 10 U/μl (Invitrogen; 18010-025), RNAse H; 2 U/μl (Invitrogen; 18021-14; 18021-071); T4-DNA polymerase; 5 U/μl (Invitrogen; 18005-025), EDTA di-sodium salt, 0.5 M solution (SIGMA; P/N E7889). Cleaning of DNA was done with the GeneChip® clean-up module in accordance with the Affymetrix protocol.

In-vitro transcription reaction. After the second-strain synthesis, the biotin-marked cRNA was generated by an in vitro transcription reaction from the cDNA sample making use of the BioArray RNA transcript labelling kit (Enzo Diagnose, Farmingdale, N.Y.) with biotin-marked CTP and UTP. The marked cRNA was cleaned with the help of RNeasy spin columns (Qiagen). 15 μg of each cRNA sample was fragmented for 35 minutes at 94° C. in fragmenting buffer (40 mM Tris-acetate, pH 8.1, 100 mM potassium acetate and 30 mM magnesium acetate) and then used for the production of 300 μl of hybridisation mixture. A biotinilysed oligonucleotide, B2, which was hybridised with control features (molecules) in the middle and on the four corners of each chip, was used as an orientation aid for the determination of the sample on the chip.

Microarray hybridisation. The Affymetrix GeneChip MG_U74Av2 was put into the GeneChip® hybridisation oven 640 (Affymetrix). Hybridisations were done for 16 hours at 45° C. and 60 RPM. The arrays were taken out of the chamber. Washing, drying and dyeing steps were done in the washing station 400 (Affymetrix) in accordance with the Affymetrix array protocol.

Microarray scanning and data recording. The hybridised arrays were scanned with a calibrated gene array scanner (Agilent) at a wavelength of 570 nm (pixel 3 μm). Each array was scanned three times. As software for data recording, the GeneChip operating software GCOS (Affymetrix) was used.

Statistical analysis. The array data were normalised, making use of scaling or per-chip normalisation, in order to balance the total or mean intensity of each array. The scale factor was set in accordance with the Affymetrix recommendation and used for generation of a microarray quality control and a data report. Features manifesting a deviating expression with a factor greater than 1.7 were regarded as being significant and recorded on an Excel spreadsheet with gene remarks and ontological data of the NetAffix™ analyses centre.

A t-test analysis was held making use of the DATA mining tool (DMT) 3.0 from Affymetrix. A stringent comparison between normal and tumour tissues was done in accordance with the consistency of the changes of the gene expression. A t-test and a ranking analysis with upward- and downward-regulated genes was held and only genes which were in a 100% concordance in the direction of change with a p value<0.05 (t-test) and an FC greater/equal 1.7/FC greater/equal −1.7 were taken into account.

RT-PCR. Specific amplimers for each gene were received from Invitrogen life technologies: Amplimer sequences (5′ to 3′), forward and correspondingly reverse were as follows: ADAM19: cgatggcggctgcatcatggc; ccaccagcttgcactggtggc; TIMP-3: gatgccccacgtgcagtacat, tgctgatgctcttgtctgggg; col17a1: ggctgagctggacggctacag, gggttcaccacgaggtcccat; Cav1: gcatcaagagcttcctgattg, ccagactgtcaaacatagatg; Bsg: aaccgggcaccatccaaacct, attgcctcttcttccccagtg; CD44: cggctccaccatcgagaagag, gttgtgggctcctgagtctga; ItgaX: ccctcaaatatgagacccacc, ggattcctgggtaaagatgac; Lamc2: gctggaaggcaggatcgagca, ttcctgccagactcaggcgca; TM4sf2: gttgattggcatgctgctggc, gcagggatagtatgtactgtg; CAP-1: cacgctcagcactgtttgcac, cacttgtagatgtaagccacc; Vtn: aagtggagcaacaggaggaga, caacattgtctggtatgccac; Ccr5: gtcaggacggtcaactttggg, gttgtagggagtccagaagag.

Quantitative RT-PCR with the light cycler (Roche Diagnostics): 10 ng of the template was mixed in 10 μl of reactions with Taq polymers (ABgene Art. no. AB 0301); 10×PCR buffer; 1 M KCl solution; 1 M Tris-HCl; pH 8.3 (RT); 60 mM MgCl2; Roth Tris (Cat. no.: 4855.2); Merck MgCL2 (Cat. no.: 8.14733.0500); Merck NaCl 37% (Cat. no. 1.00317); Sigma KCl (Cat. no.: P5405); 10 mM sNTP solution; MBI Fermentas (Cat. no.: R0181); BSA fraction V (no. G 16112-207) of PAA. SYBR Green I. (no. S-7567) Molecular Probes, USA. The cycle condition was 95° C. for 45 s, followed by 40 cycles of 1 s at 95° C. and 6 s at 72° C., with observation of the fluorescence during the annealing phase; this for its part was followed by a melting programme from 40 to 75° C. with 0.2° C./s with continuous monitoring of the fluorescence. The fluorescence quantification was calculated with the help of an integrated light cycler software 3.01 (Roche). RT-PCR (thermo-cycler): 20 mg DNA were analysed in 20 μl reactions (94° C., 1 min.; 55-58° C., 1 min.; 72° C., 2 min.). PCR reactions were carried out by means of Abgene Thermo-Start Taq (Cat. no.: AB-1908/B), 10× reaction buffer (15 mM MgCl2) and 10 mM dNTP from MBI Fermentas (Cat. no.: R0181). PCR reactions were carried out with 25-40 cycles for determination of the linear amplification range of each primer set. The quantitative determination of the bands was done by means of Kodak 1 D 3.5 network software.

Western Blot. 100 μg of proteins was separated with an SDS-PAGE gel (12% gradient) and transferred onto a PVDF membrane (NEN, cat. no. NEF 1002) for 1 h at 40 V. The membrane was blocked with 1× Roti-block (Roth, cat. no. A151.1) in 1×TBS buffer at 4° C. overnight. Proteins were detected making use of EMMPRIN (basigin): 2 μg/ml (R&D Systems, Cat. no.: MAB772) and ADAM-19: 1 mg/ml (Cerdarlane® laboratories). ADAM-19 was diluted 1:5,000. Secondary antibodies mouse/rabbit IgG (Chemicon, Cat. no.: AP 160P and Ap132P) were used with a 1:5,000 dilution. The detection of the proteins was done with a chemiluminescence reagent (NEN, Cat. no.: NEN 104) and exposed making use of the Kodak IST software 440CF.

Immunohistochemistry. Immuno-dyeing for the EpCam expression in lung cryostat sections (tissue cuts). For indirect immunofluorescence localisation of Ep-CAM positive cells in lung samples, 10 μm thick cryostat sections were attached to glass slides coated with paper adhesive, air-dried and then fixed with ice-cold methanol:acetone (1:1). The sections were rehydrated in washing buffer (1 mg bovine serum albumin (BSA)/ml phosphate buffered saline solution (PBS), pH 7.4) with rat anti-mouse-antibody Ep-CAM (BD Biosciences Pharmigen, diluted 1:1,000 in 10 mg BSA/ml PBS), incubated overnight in a moisture chamber at 4° C. and incubated in the dark at room temperature for one hour with goat anti-rat Cy-3 conjugated antibody (Chemicon, diluted 1:200 in 10 mg BSA/ml PBS) following washing steps with buffer. After subsequent washing steps, the sections were inserted into water-soluble embedded medium (Glycergel, DAKO) 4′, 6-diamidino-2-phenylindol (DAPI, Serva) for core dyeing and in Bicyclo(2,2,2,)-1,4-diazaoctane (DABCO, Sigma) as an anti-bleaching agent. Locations of the Ep-CAM resulting were made visible with a UV fluorescence microscope and documented with a digital camera. Unspecific dyeings were confirmed by leaving out the primary antibody.

Immunohistochemistry with human tissues. In the same way, immunohistological examinations were done on various human lung tumours. In this way, for example, the tumour-specific expression of epitopes EpCAM, claudin-2, CD44, CD 147 and ADAM 19 was proven both in large-celled carcinomas, squamous epithelium carcinomas and adenocarcinomas (bronchio-alveolar carcinoma, acinary and papillary adenocarcinoma and adenocarcinomas with individual mucous obstruction) of the lung.

The experiments with c-myc (over-expressed) cells/tissues were done in a corresponding way, evaluated and compared with the results of c-raf induced cells/tissues. Tables 4 and 5 show some of the results in the comparison between c-raf and c-myc (ECM change). A difference was surprisingly found for ICAM1, a surface adhesion molecule, which has only been regulated upwards in c-myc cells/tissues (FC=2.8).

### LITERATURE

1. Avruch, J. et al. Ras activation of the raf kinase: tyrosine kinase recruitment of the MAP Kinase cascade. Recent Prog Horm Res. 56, 127-55 (2001).
2. Kolch, W. Meaningful relationships: The regulation of the Ras/Raf/MEK/ERK pathway by protein interactions. Biochem J. 351, 289-305 (2000).
3. Lewis, T. S., Shapiro, P. S., Ahn, N. G. Signal transduction through MAP Kinase cascade. Adv Cancer Res. 74, 49-139 (1998).
4. Robinson, M. J., Cobb, M. H. Mitogen activated protein kinase pathways. Curr Opin Cell Biol. 9, 180-6 (1997).
5. McPhillips, F. et al. Association of c-raf expression with survival and its targeting with antisense oligonucleotides in ovarian cancer. Br J Cancer 85, 1753-8 (2001).
6. Slater, E. P. et al. c-raf controlled pathways in the protection of tumor cells from apoptosis. Int J Cancer 104, 425-32 (2003).
7. Burridge, K., Chrzanowska-Wodnicka M. Focal adhesions, contractility, and signaling. Annu Rev Cell Dev Biol 12, 463-518 (1996).
8. Gumbiner, B. M. Cell adhesion: the molecular basis of tissue architecture and morphogenesis. Cell 84, 345-57 (1996).
9. Hynes, R. O. Integrins: versatility, modulation and signaling in cell adhesion. Cell 69, 11-25 (1992). 10. Kumaki, F. et al. Expression of matrix metalloproteinases in invasive pulmonary adenocarcinoma with bronchiolalveolar component and atypical adenomatous hyperplasia. Am. J Pathol. 159, 2125-35 (2001).
11. Massova, I., Kotra, L. P., Fridman, R., Mobashery, S. Matrix metalloproteinases: structures, evolution, and diversification. FASEB J. 12, 1075-95 (1998).
12. Sato, H. et al. A matrix metalloproteinase expressed on the surface of invasive tumor cells. Nature 370, 61-5 (1994).
13. Beauvais, D. M., Burbach, B. J., Rapraeger, A. C. The syndecan-1 ectodomain regulates alphavbeta3 integrin activity in human mammary carcinoma cells. J. Cell Biol. 167, 171-81 (2004).
14. Beauvais, D. M., Rapraeger, A. C. Syndecans in tumor cell adhesion and signaling. Reprod Biol Endocrinol. 2 (1):3 (2004).
15. Kerkhoff, E. et al. Lung-targeted expression of the c-raf-1 kinase in transgenic mice exposes a novel oncogenic character of the wild-type protein. Cell Growth Differ. 11, 185-90 (2000).
16. Wang, F. et al. Heterogeneity of claudin expression by alveolar epithelial cells. Am. J. Respir. Cell Mol. Biol. 29: 62-70 (2003).
17. Turksen, K., Troy, T. C. Barriers built on claudins. J Cell Sci. 117, 2435-47 (2004).
18. Miyamori, H. et al. Claudin promotes activation of pro-matrix metalloproteinase-2 mediated by membrane type matrix metalloproteinases. J. Biol. Chem. 276, 28204-11 (2001).
19. Schafer, B. W., Heizmann, C. W. The S100 family of EF-hand calcium-binding proteins: functions and pathology. Trends Biochem. Sci. 21, 134-40 (1996).
20. Katoh, S., Zheng, Z., Oritani, K., Shimozato, T., Kincade, P. W. Glycosilation of CD44 negatively regulates its recognition of hyaluronan. J Exp Med. 182, 419-29 (1995).
21. Luscinskas, F. W.; Gimbrone, M. A. Endothelial-dependent mechanisms in chronic inflammatory leukocyte recruitment. Annu Rev Med. 47, 413-21 (1996).
22. Balzar, M., Winter, M. J., de Boder, C. J., Litvinov, S. V. The biology of the 17-1A antigen (Ep-Cam). J Mol. Med. 77, 699-712 (1999).
23. Bieritz, B. et al. Role of alpha 8 integrin in mesangial cell adhesion, migration, and proliferation. Kidney Int. 64, 119-27 (2003).
24. Scherberich, A. et al. Tetraspanin CD9 is associated with very late-acting integrins in human vascular smooth muscle cells and modulates collagen matrix reorganization. Arterioscler. Thromb. Vasc. Biol. 18, 1691-97 (1998).
25. Berditchevski, F. Complexes of tetraspanins with integrins: more than meets the eye. J Cell Sci. 114, 4143-51 (2001).
26. Thiery, J. P. Epithelial-mesenchymal transitions in tumor progression. Nat Rev Cancer 2, 442-54 (2002).
27. Muraoka, K., Nabeshima, K., Murayama, T., Biswas, C., Koono, M. Enhanced expression of a tumor-cell derived collagenase-stimulatory factor in urothelial carcinoma: its usefulness as a tumor marker for bladder cancers. Int J Cancer 55, 19-26 (1993).
28. Polette, M. et al. Tumor collagenase stimulatory factor (TCSF) expression and localization in human lung and breast cancers. J Histochem Cytochem 45, 703-9 (1997).
29. Sameshima, T. et al. Expression of emmprin (CD147), a cell surface inducer of matrix metalloproteinases, in normal human brain and gliomas. Int. J. Cancer 88, 21-7 (2000).
30. Sameshima, T. et al. Glioma cell extracellular matrix metalloproteinase inducer (EMMPRIN) (CD147) stimulates production of membrane-type matrix metalloproteinases and activate gelatinase A in co cultures with brain-derived fibroblasts. Cancer Lett. 157, 177-84 (2000).
31. Jiang, J. L. et al. The involvement of HAb18G/CD147 in regulation of store-operated calcium entry and metastasis of human hepatoma cell. J. Biol. Chem. 276, 46870-7 (2001).
32. Bordador, L. C. et al. Expression of emmprin by oral squamous cell carcinoma. Int. J. Cancer 85, 347-52 (2000).
33. Van den Oord, J. J., Paemen, L., Opdenakker, G., de Wolf-Peeters, C. Expression of gelatinase B and the extracellular matrix metalloproteinase inducer EMMPRIN in benign and malignant pigment cell lesions of the skin. Am J Pathol 151, 665-70 (1997).
34. Kanekura, T., Chen, X., Kanzaki, T. Basigin (CD147) is expressed on melanoma cells and induces tumor cell invasion by stimulating production of matrix metalloproteinases by fibroblasts. Int. J. Cancer 99, 520-8 (2002).
35. Marieb, E. A. et al. Emmprin promotes anchorage-independent growth in human mammary carcinoma cells by stimulating hyaluronan production. Cancer Res. 64, 1229-32 (2004).
36. Tang, W., Chang, S. B., Hemler, M. E. Links between CD147 Function, Glycosylation, and Caveolin-1. Mol Biol Cell 15, 4043-50 (2004).
37. Chesneau, V. et al. Catalytic properties of ADAM-19. J Biol. Chem. 278, 22331-40 (2003).
38. Franzke, C. W., Tasanen, K., Borradori, L., Huotari, V., Bruckner-Tuderman, L. Shedding of collagen XVII/BP180: structural motifs influence cleavage from cell surface. J Biol. Chem. 279, 24521-9 (2004).
39. Franzke, C. W., Tasanen, K., Schumann, H., Bruckner-Tuderman, L. Collagenous transmembrane proteins: collagen XVII as a prototype. Matrix Biol. 22, 299-309 (2003).
40. Franzke, C. W. et al. Transmembrane collagen XVII, an epithelial adhesion protein, is shed from the cell surface by ADAMs. EMBO J. 21, 5026-35 (2002).
41. Tasanen, K., Tunggal, L., Chometon, G., Bruckner-Tuderman, L., Aumailley, M. Keratinocytes from patients lacking collagen XVII display a migratory phenotype. Am J. Pathol. 164 2027-38 (2004).
42. Karan, D. et al. Expression of ADAMs (a disintegrin and metalloproteinases) and TIMP-3 in human prostatic adenocarcinomas. Int. J. Oncol. 23, 1365-71 (2003).
43. Gill, S. E., Pape, M. C., Khokha, R., Watson, A. J., Leco, K. J. A null mutation for tissue inhibitor of metalloproteinase-3 (Timp-3) impairs murine bronchiole branching morphogenesis. Dev Biol. 261, 313-23 (2003).
44. Baker, A. H., George, S. J., Zaltsman, A. B., Murphy, G., Newby, A. C. Inhibition of invasion and induction of apoptotic cell death of cancer cell lines by overexpression of TIPMP-3. Br J Cancer 79, 1347-55 (1999).
45. Loetscher, P. et al. CCR5 is characteristic of Th1 lymphocytes. Nature 391, 344-5 (1998).
46. Wen, T., Bukczynski, J., Watts, T. H. 4-1 BB ligand-mediated costimulation of human T cells induces CD4 and CD8 T cell expansion, cytokine production, and the development of cytolytic effector function. J. Immunol. 168, 4897-906 (2002).

### LEGEND ON THE TABLES

Table 1: Over-expressed genes relative to the ECM re-modelling in non-cellular lung carcinomas
Table 2a: Repressed genes relative to the ECM re-modelling of adhesion molecules in non-small-cell lung carcinomas
Table 2b: Differentially expressed genes relative to the cell-induced immunity and inflammation in non-cellular lung carcinomas
Table 3: Confirmation of the differentially expressed genes by means of qRT-PCR.
Table 4: Induced genes relative to the ECM re-modelling in non-cellular lung carcinomas
Table 5: Repressed genes relative to the ECM re-modelling of adhesion molecules in non-small-cell lung carcinomas

TABLE 1
Overexpressed genes related with ECM remodelling in non-cell lung carcinoma

| Affymetrix | | | | |
|---|---|---|---|---|
| probe set ID | Gene symbol | Gene Description | Fold change | P-value |
| 103554_at | ADAM19 | a Disintegrin and metalloproteinase 19 | 3.17 | <0.01 |
| 95338_s_at | Mmp12 | Metalloproteinase 12 | 2.37 | <0.01 |
| 101881_g_at | Col18a1 | Procollagen, type XVIII alpha1 | 2.5 | <0.01 |
| 99637_at | Col15a1 | Procollagen, type XV alpha1 | 4.95 | <0.01 |
| 103005_s_at | CD44 | CD 44 | 3.97 | <0.01 |
| 101078_at | Bsg | Basigin | 2.8 | <0.01 |
| 102353_at | Itgb2 | Integrin beta 2 | 3.99 | <0.01 |
| 104308_at | Itgax | Integrin alpha 10 | 3.25 | <0.01 |
| 100428_at | Lamc2 | Laminin gamma 2 | 4.21 | <0.01 |
| 92759_at | Lamb3 | Laminin beta 3 | 3.28 | <0.01 |
| 104407_at | Alcam | activated leukocyte cell adhesion molecule | 1.81 | <0.01 |
| 93934_at | Cldn2 | Claudin 2* | 20.26 | 0.02 |
| 94493_at | Cldn3 | Claudin 3 | 2.41 | <0.01 |
| 99561 _f_at | Cldn7 | Claudin 7 | 2.07 | <0.01 |
| 94270_at | Krt1-18 | Keratin complex 1, acidic gene 18 | 2.67 | <0.01 |
| 101009_at | Krt2-8 | Keratin complex 2, basic gene 8 | 1.71 | <0.01 |
| 99582_at | tacstd1 | tumor associated calcium signal transducer 1 | 1.77 | <0.01 |
| 160651_at | tacstd2 | tumor associated calcium signal transducer 2 | 2.37 | <0.01 |
| 95453_f_at | S100 a1 | S100 calcium binding protein A1 | 1.99 | <0.01 |
| 98600_at | S100 a11 | S100 calcium binding protein A11 | 1.76 | <0.01 |

Table 1: Overexpressed genes (>1.7-fold, P < 0.1) related with ECM remodelling in lung adenocarcinoma induced by c-raf in mice. Fold change was calculated by Affymetrix software (DMT 3.0).
*Genes detected as “present” only in lung adenocarcinoma.

TABLE 2a
Repressed genes related with remodelling of adhesion
molecules in non- small cell lung carcinoma

| Affymetrix | | | | |
|---|---|---|---|---|
| probe set ID | Gene symbol | Gene Description | Fold change | P-value |
| 94963_at | Vcl | Vinculin | −1.75 | <0.01 |
| 98549_at | Vtn | Vitronectin* | −4.24 | 0.02 |
| 160280_at | Cav1 | Caveolin 1 | −2.49 | <0.01 |
| 160610_at | Pcdha4 | Procadherin alpha 4* | −9.85 | <0.01 |
| 104083_at | Cdh5 | Cadherin 5 | −3.54 | <0.01 |
| 102852_at | Cdh2 | Cadherin 2* | −2.58 | <0.01 |
| 104743_at | Cdh13 | Cadherin 13* | −4.38 | 0.03 |
| 101948_at | Lamb1-1 | Laminin beta 1, subunit 1 | −2.95 | <0.01 |
| 92366_at | Lama2 | Laminin alpha 2 | −2.33 | <0.01 |
| 104587_at | Lama4 | Laminin alpha 4 | −2.4 | <0.01 |
| 100313_at | Itga8 | Integrin alpha 8* | −5.9 | <0.01 |
| 100134_at | Eng | Endoglin | −3.6 | <0.01 |
| 161907_s_at | Tnxb | Tenascin XB | −2.7 | 0.02 |
| 103721_at | Npnt | Nephronectin | −1.9 | <0.01 |
| 96742_at | Dpt | Dermatoponin | −4.15 | <0.01 |
| 101499_at | Ilk | Integrin linked kinase | −1.71 | <0.01 |
| 102261_f_at | Col13a1 | Procollagen, type XIII alpha1* | −6.74 | 0.02 |
| 160594_at | Col17a1 | Procollagen, type XVII alpha1* | −4.6 | 0.04 |
| 94085_at | Prg | Proteoglycan, secretory granule | −2.36 | <0.01 |
| 104375_at | Spock2 | Sparc/osteonectin, cwcv and Kazal-like | −2.78 | <0.01 |
| | | domains, proteoglycan 2 | | |
| 95104_at | Sdc2 | Syndecan 2 | −2.32 | <0.01 |
| 94112_at | Tnfsf10 | tumor necrosis factor superfamily, member 10 | −2.16 | <0.01 |
| 97832_at | CD97 | CD 97 | −3.83 | <0.01 |
| 103611_at | CD47 | CD 47 | −1.7 | <0.01 |
| 99053_at | Icam2 | Intracellular adhesion molecule 2* | −6.69 | <0.01 |
| 104516_at | Cldn5 | Claudin 5 | −5 | <0.01 |
| 160458_at | Mcam | Melanoma cell adhesion molecule | −1.9 | <0.01 |
| 160519_at | TIMP3 | tissue inhibitor metalloproteinase 3 | −2.76 | <0.01 |
| 100447_at | Hyal2 | Hyaluronidase 2 | −3.16 | <0.01 |
| 93887_at | Mpdz | Multiple PDZ domain protein | −3.51 | <0.01 |
| 97977_at | Ntn1 | Netrin | −2.3 | <0.01 |
| 160583_at | Xlkd1 | Extracellular link domain containing 1 | −8.99 | <0.01 |
| 103644_at | Dpep1 | Dipeptidase1 | −5.08 | <0.01 |
| 97984_i_at | Kns2 | Kinesin 2 | −1.76 | <0.01 |
| 97976_at | Ktn1 | Kinectin 1 | −1.86 | <0.01 |
| 97358_at | Lphn1 | Latrophilin | −1.74 | <0.01 |
| 101095_at | Mfap2 | Microfibrillar associated protein 2 | −2.45 | <0.01 |
| 161962_f_at | Mfap4 | Microfibrillar associated protein 4 | −4.51 | <0.01 |
| 93721_at | Cap1 | CAP adenylate cyclase, associated protein 1* | −8.84 | <0.01 |
| 104121_at | Jup | Junction plakoglobin | −1.7 | <0.01 |
| 96774_at | Plekhc1 | Pleckstrin homology domain containing family | −2.05 | <0.01 |
| | | (with FERM domain member 1) | | |
| 97497_at | Notch1 | Notch 1 | −2.21 | <0.01 |
| 104188_at | Notch2 | Notch 2 | −1.96 | <0.01 |
| 92652_at | Notch4 | Notch 4 | −5.49 | <0.01 |
| 98349_at | Il6st | Interleukine 6 signal transducer | −2.43 | <0.01 |
| 101522_at | TM4SF1 | Transmembrane 4 superfamily 1* | −2.35 | <0.01 |
| 93326_at | TM4SF2 | Transmembrane 4 superfamily 2 | −4.66 | <0.01 |
| 92555_at | TM4SF6 | Transmembrane 4 superfamily 6 | −2.98 | <0.01 |
| 160678_at | TM4SF12 | Transmembrane 4 superfamily 12 | −4.75 | <0.01 |
| 95120_at | TM4SF13 | Transmembrane 4 superfamily 13 | −3.43 | <0.01 |
| 102720_at | TEK | endothelial specific receptor tyrosine kinase | −6.41 | <0.01 |
| 104697_at | Rhoj | ras homolog gene family, member J | −2.49 | <0.01 |

Table 2a: repressed genes (≦1.7-fold, P < 0.1) related with remodelling of cell adhesion in lung adenocarcinoma induced by c-raf in mice. Fold change was calculated by Affymetrix software (DMT 3.0).
*Genes detected as “absent” in lung adenocarcinomas.

TABLE 2b
Differentially expressed genes related with cell-mediated
immunity and inflamation in non-cell lung carcinoma.

| Affymetrix | | | | |
|---|---|---|---|---|
| probe set ID | Gene symbol | Gene Description | Fold change | P-value |
| 97444_at | Ifi30 | Interferon gamma inducible protein 30 | 4.67 | <0.01 |
| 92415_at | Tnfsf9 | Tumor necrosis factor superfamily 9* | 12.52 | <0.01 |
| 94747_at | Csf2rb1 | Colony stimulating factor 2, receptor beta 1 | 4.28 | <0.01 |
| 103210_at | Csf2rb2 | Colony stimulating factor 2, receptor beta 2 | 2.43 | <0.01 |
| 99330_at | Csf2ra | Colony stimulating factor 2, receptor alpha | 3.58 | <0.01 |
| 161968_f_at | Ccr5 | Chemokine (C-C motif) receptor 5 | 4.82 | <0.01 |
| 92849_at | Ccl6 | Chemokine (C-C motif) ligand 6 | 7.63 | <0.01 |
| 103012_at | Ccl21b | Chemokine (C-C motif) ligand 21b | 1.92 | <0.01 |
| 98088_at | CD14 | CD 14 | 2.83 | <0.01 |
| 103005_s_at | CD44 | CD 44 | 3.97 | <0.01 |
| 93092_at | H2-DMa | Histocompatibility 2, class II, locus DMa | 1.92 | <0.01 |
| 98035_g_at | H2-DMb1 | Histocompatibility 2, class II, locus DMb1 | 4.54 | <0.01 |
| 100998_at | H2-Ab1 | Histocompatibility 2, class II antigen A, beta | 3.19 | <0.01 |
| 92866_at | H2-Aa | Histocompatibility 2, class II antigen A, alpha | 2.66 | <0.01 |

Table 2b: up-regulated genes related with cell-mediated immunity (>1.7-fold, P < 0.1). Fold change was calculated by Affymetrix software (DMT 3.0).
*Genes detected as “absent” in normal lung tissues.

TABLE 3
Confirmed differentially expressed genes by qRT-PCR

| | | Method | | | | |
|---|---|---|---|---|---|---|
| | | Light cycler | Thermocycler | | | PCR |
| | Probe Set ID | FC | Tm | FC | Confirmed | (bp) |
| repressed genes in | | | | | | |
| lung adenocarcinoma | | | | | | |
| TIMP-3 | 160519_at | −1.87 ± 0.42 | 87° | −2.23 ± 0.76 | + | 373 |
| Caveolin-1 | 160280 at | −2.46 ± 0.71 | 85° | −2.67 ± 0.98 | + | 326 |
| CAP-1 | 93721_at | — | | −3.59 ± 1.57 | + | 289 |
| TM4 sf 2 | 93326_at | −4.46 ± 0.61 | 86° | −4.88 ± 1.39 | + | 180 |
| Col17a1 | 160594_at | — | | −7.59 ± 2.45 | + | 295 |
| Vitronectin | 98549_at | — | | −4.86 ± 1.12 | + | 375 |
| overexpressed genes in | | | | | | |
| lung adenocarcinoma | | | | | | |
| ADAM-19 | 103554_at | — | | +2.65 ± 1.01 | + | 307 |
| Basigin | 101078_at | +2.34 ± 0.82 | 86° | +2.84 ± 1.78 | + | 391 |
| CD44 | 103005_s_at | +2.23 | 87° | +2.61 ± 1.23 | + | 230 |
| ItgaX | 104308_at | — | | +2.82 ± 0.54 | + | 154 |
| Lamc2 | 100428_at | — | | +4.61 ± 1.53 | + | 254 |
| Ccr5 | 161968_f_at | +5.10 ± 1.76 | 86° | +4.16 ± 2.22 | + | 320 |

Fold change (FC) determined by RT-PCR. Comparison of tumoral tissues from c-raf-1 lung adenocarcinomas (n = 3) with normal lung tissues used as controls (n = 3). Quantitation of the bands was performed with the Kodak 1D 3.5 Network software (thermocycler) and fluorescence quantification (LightCycler) was performed with the aid of built (LightCycler software 3.0.1).

TABLE 4
Induced genes relative to the ECM re-modelling in non-cellular lung carcinomas

| Affymetrix | | | | |
|---|---|---|---|---|
| probe set ID | gene-symbol | gene-description | c-raf | c-myc |
| 103554_at | ADAM19 | a Disintegrin and metalloproteinase 19 | 3.17 | 2.3 |
| 95338_s_at | Mmp12 | Metalloproteinase 12 | 2.37 | <0.01 |
| 101881_g_at | Col18a1 | Procollagen, type XVIII alpha1 | 2.5 | <0.01 |
| 99637_at | Col15a1 | Procollagen, type XV alpha1 | 4.95 | 4.6 |
| 103005_s_at | CD44 | CD 44 | 3.97 | <0.01 |
| 101078_at | Bsg | Basigin | 2.8 | <0.01 |
| 102353_at | Itgb2 | Integrin beta 2 | 3.99 | <0.01 |
| 104308_at | Itgax | Integrin alpha 10 | 3.25 | <0.01 |
| 100428_at | Lamc2 | Laminin gamma 2 | 4.21 | <0.01 |
| 92759_at | Lamb3 | Laminin beta 3 | 3.28 | <0.01 |
| 104407_at | Alcam | activated leukocyte cell adhesion molecule | 1.81 | <0.01 |
| 93934_at | Cldn2 | Claudin 2* | 20.26 | 43.5 |
| 94493_at | Cldn3 | Claudin 3 | 2.41 | <0.01 |
| 99561_f_at | Cldn7 | Claudin 7 | 2.07 | <0.01 |
| 94270_at | Krt1-18 | Keratin complex 1, acidic gene 18 | 2.67 | 3.6 |
| 101009_at | Krt2-8 | Keratin complex 2, basic gene 8 | 1.71 | <0.01 |
| 99582_at | tacstd1 | tumor associated calcium signal transducer | 1.77 | <0.01 |
| | | 1 | | |
| 160651_at | tacstd2 | tumor associated calcium signal transducer | 2.37 | <0.01 |
| | | 2 | | |
| 95453_f_at | S100 a1 | S100 calcium binding protein A1 | 1.99 | <0.01 |
| 98600_at | S100 a11 | S100 calcium binding protein A11 | 1.76 | <0.01 |

Table 4:: Overexopressed genes (>1.7-fold, P < 0.1) genes relative to the ECM re-modelling in
lung adenocarcinomas induced by c-raf and c-myc mices, respectively. The fold change was determined by Affymetrix Software (DMT 3.0).
*genes, which are detected as present only in lung adenocarcinomas.

TABLE 5
Repressed genes relative to the ECM re-modelling of
adhesion molecules in non-small-cell lung carcinomas

| Affymetrix | | | | |
|---|---|---|---|---|
| probe set ID | Gene symbol | gene description | c-raf | c-myc |
| 94963_at | Vcl | Vinculin | −1.75 | <0.01 |
| 98549_at | Vtn | Vitronectin* | −4.24 | −6.6 |
| 160280_at | Cav1 | Caveolin 1 | −2.49 | −6.5 |
| 160610_at | Pcdha4 | Protocadherin alpha 4* | −9.85 | −9.9 |
| 104083_at | Cdh5 | Cadherin 5 | −3.54 | −10 |
| 102852_at | Cdh2 | Cadherin 2* | −2.58 | <0.01 |
| 104743_at | Cdh13 | Cadherin 13* | −4.38 | 0.03 |
| 101948_at | Lamb1-1 | Laminin beta 1, subunit 1 | −2.95 | <0.01 |
| 92366_at | Lama2 | Laminin alpha 2 | −2.33 | <0.01 |
| 104587_at | Lama4 | Laminin alpha 4 | −2.4 | −2.91 |
| 100313_at | Itga8 | Integrin alpha 8* | −5.9 | −8.8 |
| 100134_at | Eng | Endoglin | −3.6 | −7.6 |
| 161907_s_at | Tnxb | Tenascin XB | −2.7 | 0.02 |
| 103721_at | Npnt | Nephronectin | −1.9 | <0.01 |
| 96742_at | Dpt | Dermatoponin | −4.15 | −17 |
| 101499_at | Ilk | Integrin linked kinase | −1.71 | <0.01 |
| 102261_f_at | Col13a1 | Procollagen, type XIII alpha1* | −6.74 | −17 |
| 160594_at | Col17a1 | Procollagen, type XVII alpha1* | −4.6 | 0.04 |
| 94085_at | Prg | Proteoglycan, secretory granule | −2.36 | −5 |
| 104375_at | Spock2 | Sparc/osteonectin, cwcv and Kazal-like | −2.78 | −22 |
| | | domains, proteoglycan 2 | | |
| 95104_at | Sdc2 | Syndecan 2 | −2.32 | <0.01 |
| 94112_at | Tnfsf10 | tumor necrosis factor superfamily, member | −2.16 | <0.01 |
| | | 10 | | |
| 97832_at | CD97 | CD 97 | −3.83 | −9.7 |
| 103611_at | CD47 | CD 47 | −1.7 | <0.01 |
| 99053_at | Icam2 | Intracellular adhesion molecule 2* | −6.69 | −48 |
| 104516_at | Cldn5 | Claudin 5 | −5 | −17.8 |
| 160458_at | Mcam | Melanoma cell adhesion molecule | −1.9 | <0.01 |
| 160519_at | TIMP3 | tissue inhibitor metalloproteinase 3 | −2.76 | <0.01 |
| 100447_at | Hyal2 | Hyaluronidase 2 | −3.16 | <0.01 |
| 93887_at | Mpdz | Multiple PDZ domain protein | −3.51 | <0.01 |
| 97977_at | Ntn1 | Netrin | −2.3 | <0.01 |
| 160583_at | Xlkd1 | Extracellular link domain containing 1 | −8.99 | −28 |
| 103644_at | Dpep1 | Dipeptidase1 | −5.08 | −4.9 |
| 97984_i_at | Kns2 | Kinesin 2 | −1.76 | <0.01 |
| 97976_at | Ktn1 | Kinectin 1 | −1.86 | <0.01 |
| 97358_at | Lphn1 | Latrophilin | −1.74 | <0.01 |
| 101095_at | Mfap2 | Microfibrillar associated protein 2 | −2.45 | −5.8 |
| 161962_f_at | Mfap4 | Microfibrillar associated protein 4 | −4.51 | <0.01 |
| 93721_at | Cap1 | CAP adenylate cyclase, associated protein | −8.84 | <0.01 |
| | | 1* | | |
| 104121_at | Jup | Junction plakoglobin | −1.7 | <0.01 |
| 96774_at | Plekhc1 | Pleckstrin homology domain containing | −2.05 | <0.01 |
| | | family | | |
| | | (with FERM domain member 1) | | |
| 97497_at | Notch1 | Notch 1 | −2.21 | <0.01 |
| 104188_at | Notch2 | Notch 2 | −1.96 | <0.01 |
| 92652_at | Notch4 | Notch 4 | −5.49 | −5.81 |
| 98349_at | Il6st | Interleukine 6 signal transducer | −2.43 | −4.3 |
| 101522_at | TM4SF1 | Transmembrane 4 superfamily 1* | −2.35 | <0.01 |
| 93326_at | TM4SF2 | Transmembrane 4 superfamily 2 | −4.66 | −8.5 |
| 92555_at | TM4SF6 | Transmembrane 4 superfamily 6 | −2.98 | <0.01 |
| 160678_at | TM4SF12 | Transmembrane 4 superfamily 12 | −4.75 | <0.01 |
| 95120_at | TM4SF13 | Transmembrane 4 superfamily 13 | −3.43 | −13 |
| 102720_at | TEK | endothelial specific receptor tyrosine | −6.41 | −26 |
| | | kinase | | |
| 104697_at | Rhoj | ras homolog gene family, member J | −2.49 | <0.01 |

Only in c-myc

| | | | |
|---|---|---|---|
| 92559_at | Vcam | Vascular cell adhesion molecule | −4.08 |
| 97790_s_at | Lama3 | Laminin, alpha 3 | −5.2 |
| 101650_at | Pcdha6 | Protocadherin alpha 6 | −9 |
| 98833_at | Mmp3 | Matrix metalloproteinase 3 | −6.2 |
| 96886_at | Stab1 | Stabilin 1 | −3.4 |
| 96825_at | Tenc1 | Tensin like C1 domain containing phosphatase | −6.8 |
| 106319_at | Sparcl1 | SPARC-like 1 | −5.4 |

Table 5: Repressed genes (≦1.7-fold, P < 0.1) relative to the ECM re-modelling of adhesion molecules in adenocarcinomas, induced by c-raf and c-myc respectively in mice. The fold change was determined by Affymetrix Software (DMT 3.0).
*genes, which are detected as absent in

### LEGEND ON THE ILLUSTRATIONS

FIG. 1: Immunohistochemistry of Ep-Cam in lung adenocarcinomas

FIG. 2: Western Blots of basigin and ADAM19

FIG. 3: Schematic model with some of the transcription changes changed by c-raf-1 in the ECM restructuring and cell adhesion

### EXPLANATIONS ON THE ILLUSTRATIONS

### FIG. 1:

### Immunohistochemistry of Ep-Cam

(a) Dapi dyeing in nuclei and Ep-Cam dyeing of lung adenocarcinomas
(b) Ep-Cam dyeing in tumour foci

### FIG. 2:

### Western Blots of Basigin and ADAM19

Basigin was analysed making use of a monoclonal anti-mouse EMMPRIN antibody. All the bands show basigin at 55 KDa, but basigin is increased in c-raf-1 lung tumours. ADAM-19 was analysed by use of a rabbit anti-human ADAM-19 antibody against the N-terminus of furin-activated human ADAM-19. Mouse ADAM-19 is identified with a band of 60 KDa which is only detected in lung tumour tissues. Tracks 1.3 correspond to c-raf-1 lung tumours (n=3) of transgenic mice; tracks 4-5 are controls (non-transgenic lung tissue). L: protein conductor. For the individual tracks, 100 μg of protein extracts of lung controls and lung tumours are used.

### FIG. 3:

Schematic model, which portrays some of the most highly significantly changed transcript changes in the ECM restructuring and cell adhesion in lung adenocarcinomas induced by c-raf-1 in mice.

We examined the expression of genes coding for ECM and cell adhesion molecules in lung adenocarcinomas by microarray, qRT-PCR, immunohistochemistry ad Western immuno-blotting techniques.

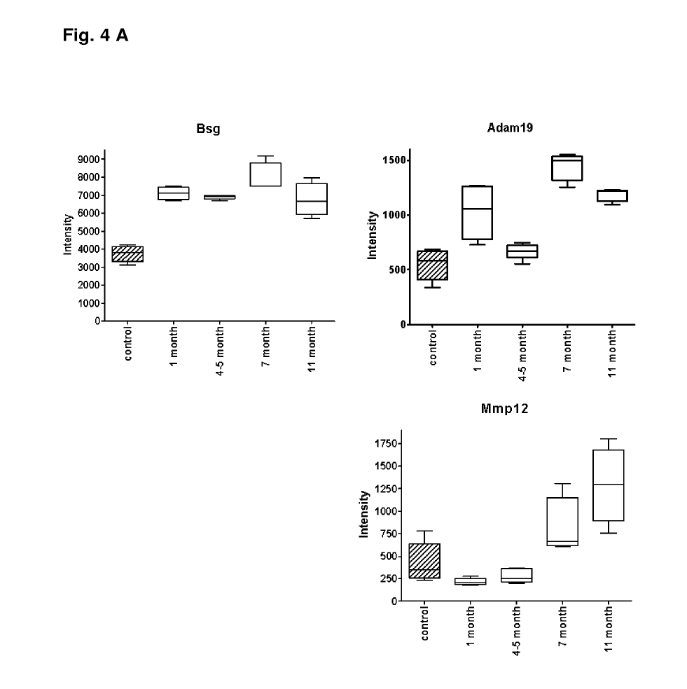

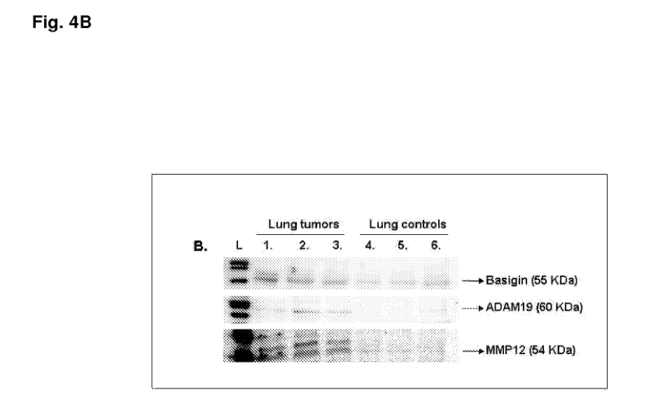

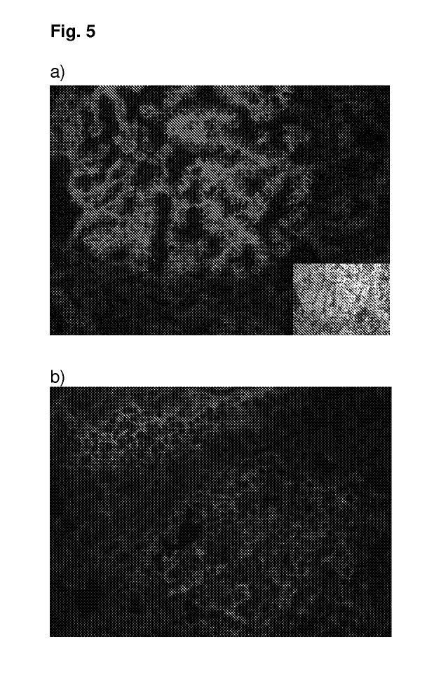

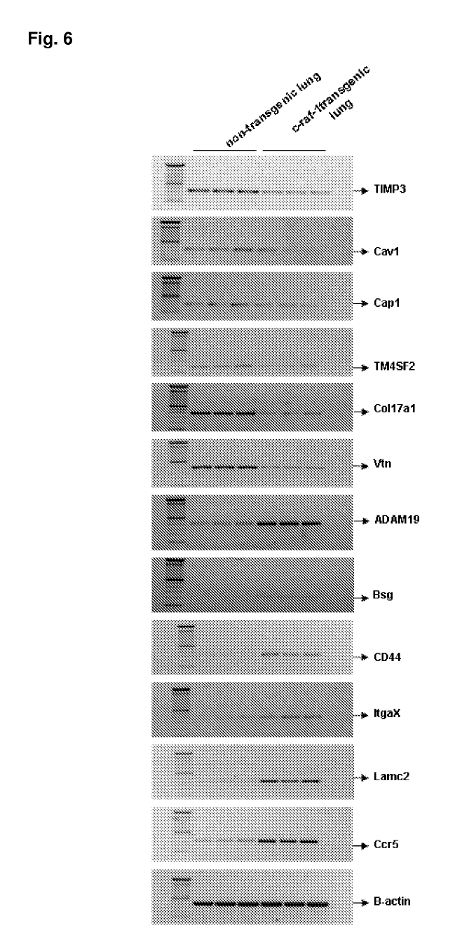

We used qRT-PCR (Table 7) in order to confirm the results obtained by means of GeneChip® microarrays. FIG. 6 shows the results of 12 of the genes described, whereas FIGS. 4B and 5 portray the expression of selected proteins. In fact, we established a good correspondence amongst the methods used.

An extremely remarkable effect was the induction of MMP12 and ADAM19 (Table 6, FIG. 6A). Alongside CD44, we found basigin induced, an extra-cellular matrix metalloproteinase, whereas their inhibitor caveolin-1 was repressed. Apart from this, the induction of Mmp12 and ADAM19, a new kind of disintegrin metalloproteinase, correlated with the inhibition of Timp-3, an inhibitor of proteins of the ADAM family.

These transcriptional changes contributed to an increase of the proteolytic activity in malignant lung tumours.

In addition, we observed changed expression of CAM molecules, including cadherin 2, 5, 13, procadherin alpha 4, procollagen Type XIII and XVII, laminins, integrin alpha 8, Icam2, tetrapsins, Ep-CAM and claudins. We show re-designed CAM and ECM molecules as characteristics of invasive lung adenocarcinomas and therefore identified new possibilities of mechanism-based therapies for lung cancer.

We observed the expression of cell adhesion and ECM-coding genes in histologically unambiguously defined adenocarcinomas of the lung and compared the results with normal, non-transgenic lung tissues.

We then held microarray studies at various times of the development of lung tumours.

To start with, we carried out a hierarchic cluster analysis and, as specified in the method part, searched for genes, the expression of which is done together as a group. The algorithm used was able to make a clear distinction between the various lung tumours. This indicated that expression profiling would enable us to distinguish between the various lung tumours and separate 11-month-old tumours from young ones.

We identified basigin or EMMPRIN (extra-cellular matrix metalloproteinase inductor) as being increased to a great extent in all the lung adenocarcinomas examined. In particular, we observed the repression of caveolin (Cav) and induction of the matrix metalloproteinase-12 (MMp12) and disintegrin- and metalloproteinase 19 (ADAM19) as a result of the Bsg induction.

The extra-cellular matrix metalloproteinase inductor Bsg, ADAM19 and MMP12 were further examined on the protein level and we confirmed all gene products as significantly increased in lung tumours as shown in FIG. 4B. Over and above this, the degree of MMP-12 and ADAM-19 expression increases with the progressive stage of the tumour growth and appears connected with the epithelial de-differentiation in lung tumours.

Further, we discovered the selective expression of collagens of the basal membrane and collagen-like transmembrane proteins which are extensively expressed and which are involved in the cell adhesion and the epithelial-mesenchymal interactions during the morphogenesis.

We discovered the selective expression of claudins (Cldn) as a characteristic of lung adenocarcinomas. The expression of claudin-2 was completely missing in normal lung tissue, but was strongly induced in intermediary and later stages of the lung tumour growth. In addition, expression of claudin 3 and 7 was increased, whereas claudin 5 was repressed in lung tumour tissues.

Further molecules which we are identified and are suitable for the classification of a lung tumour contain keratin 1-18. Calcium-binding proteins (S100A1 and S100A11) were likewise induced.

In addition, we discovered that CD44 is induced from the 1-month, early, intermediary and progressed stage of lung tumours.

Expression of genes for cell adhesion and cell-cell interaction modules was likewise induced, e.g. of integrins (Itgb2 and Itgax), procollagen XV (Col15a1) and laminins (Lamb3 and Lamc2).

Over-expression of tumour-associated calcium signal transductor (tacstd) or (Ep-CAM) was a further important discovery of our examination, both with regard to its function as an adhesion molecule and also for its activity as a therapeutic target.

Histology. Paraffin blocks were sub-divided into 3 to 5 millimetre thick slices and dyed with haematoxylin and eosin (H and E), haematoxylin (H) alone and PAS for light-microscopy evaluation.

Extraction of total RNA. RNA extraction and purifications were done by means of RNeasy Midi Kit (Qiagen, Cat. no.: 75144) in accordance with the manufacturer's instructions.

Microarray analysis. 10 μg of total RNA was used as the starting material in order to produce cDNA. The synthesis of double-stranded cDNA was done with the GeneChip® one-cycle cDNA kit (Affymetrix). Purification of double-stranded cDNA was done by use of the GeneChip® sample cleanup module (Affymetrix).

In-vitro transcription was done with the GeneChip® IVT marking kit (Affymetrix). The total amount of the reaction product was cleaned with the GeneChip® sample cleanup module (Affymetrix). Purified cRNA was determined quantitatively and checked for its quality making use of the NanoDrop ND-1000 and the Agilent 2100 bio-analyser.

Cleaned cRNA was split into fragments of 35-200 bases by metal-induced hydrolysis. The degree of fragmenting and the length distribution of the fragmented biotinilysed cRNA was checked by capillary electrophoresis by means of the Agilent 2100 bio-analyser. 10 μg of the fragmented biotinilysed cRNA was hybridised on the GeneChip® genome array in accordance with the manufacturer's instructions. The hybridisation was done in the GeneChip® hybridisation oven 640 (Affymetrix) for 16 hours at 60 RPM and 45° C. Washing and dyeing of the array were done on the Gen Chip® fluidics station 450 (Affymetrix) in accordance with the manufacturer's instructions.

The antibody signal amplification, washing and dyeing protocol (Affymetrix) was used in order to dye arrays with streptavidin r-phycoerythrin (SAPE; Invitrogen). To reinforce the dyeing, SAPE solution with a biotinilysed anti-streptavidin antibody (Vector Laboratories, CA) was added twice between the dyeing step. The arrays were scanned by using a GeneChip® scanner 3000. Scanned image files were visually examined for artefacts and then analysed, each picture being scaled to the same target value for comparison between the chips. The GeneChip® operating system (GCOS) was used in order to control the fluidics station and the scanner in order to detect the data of test arrays and to analyse data on the hybridisation intensity. Standard parameters, which are provided in the Affymetrix data analysis software package, were used for the analysis.

Statistical analysis. Array data were normalised by using scaling or per-chip normalisation in order to set the overall or average intensity of each array roughly identically. The standardisation factor (SF) was according to the setting SF=3 recommended by Affymetrix and the TGT value was 250 and was used in order to generate a microarray quality control and a data report. Features which manifest significantly differing expression were regarded as being significant and transferred to an Excel spreadsheet calculation with a gene remark and ontological data, which were received from the NetAffx™ analysis centre. A t-test analysis was held by using data mining tool (DMT) 3.0 from Affymetrix. A stringent comparison between the normal and tumour tissues was held in accordance with the consistency of changes of the gene expression. A t-test and a classification analysis with genes regulated upwards or downwards was held and merely genes manifesting a 100% correspondence in the direction of change with a P value<0.05 (t-test) were taken into account.

Cluster analysis. The hierarchic cluster analysis was done by means of the cluster tool, which is an integral part of the ArrayTrack 3.1.5 software. (http://www.fda.gov/nctr/science/centers/toxicoinformatics/ArrayTrack/). The hierarchic clustering was calculated by making use of the Pearson correlation as a metric distance and Ward's method for the set-up of the cluster tree.

qRT-PCR. Specific amplifiers for each gene were received from Invitrogen Life Technologies; the amplimer sequences (5′ to 3′) forwards and reverse were as follows: ADAM19: cgatggcggctgcatcatggc; ccaccagcttgcactggtggc; TIMP-3: gatgccccacgtgcagtacat, tgctgatgctcttgtctgggg; col17a1: ggctgagctggacggctacag, gggttcaccacgaggtcccat; Cav1: gcatcaagagcttcctgattg, ccagactgtcaaacatagatg; Bsg: aaccgggcaccatccaaacct, attgcctcttcttccccagtg; CD44: cggctccaccatcgagaagag, gttgtgggctcctgagtctga; ItgaX: ccctcaaatatgagacccacc, ggattcctgggtaaagatgac; Lamc2: gctggaaggcaggatcgagca, ttcctgccagactcaggcgca; TM4sf2: gttgattggcatgctgctggc, gcagggatagtatgtactgtg; CAP-1: cacgctcagcactgtttgcac, cacttgtagatgtaagccacc; Vtn: aagtggagcaacaggaggaga, caacattgtctggtatgccac; Ccr5: gtcaggacggtcaactttggg, gttgtagggagtccagaagag.

RT-PCR (thermo-cycler): 20 mg DNA were analysed in 20 μl reactions (94° C., 1 min.; 55-58° C., 1 min.; 72° C., 2 min.). PCR reactions were carried out by means of ABgene Thermo-Start Taq (Cat. no.: AB-1908/B), 10× reaction buffer (15 mM MgCl2) and 10 mM dNTP from MBI Fermentas (Cat. no.: R0181). PCR reactions were carried out with 25-40 cycles for determination of the linear amplification range of each primer set. The quantitative determination of the bands was done by means of Kodak 1D 3.5 network software.

Western Blot. 100 μg of proteins was separated with an SDS-PAGE with a gradient of 12% and transferred onto a PVDF membrane (NEN, cat. no. NEF 1002) for 1 h at 40 V. The membrane was blocked with 1× Roti-block (Roth, cat. no. A151.1) in 1×TBS buffer at 4° C. overnight. Proteins were detected making use of antibodies against c-raf (Santa Cruz Biotechnologies, Cat. no.: Sc 133); EMMPRIN or basigin (R&D Systems, Cat. no.: MAB772) and ADAM-19 (Cerdarlane® laboratories). ADAM-19 was diluted 1:5,000. Secondary antibodies mouse/rabbit IgG (Chemicon, Cat. no.: AP 160P and Ap132P) were used with a 1:5,000 dilution. The detection of the proteins was done with a chemiluminescence reagent (NEN, Cat. no.: NEN 104) and exposed making use of the Kodak IST software 440CF.

Immunohistochemistry. 10 μm thick cryostat sections were attached to glass slides coated with paper adhesive, air-dried and then fixed with ice-cold methanol:acetone (1:1). The sections were rehydrated in washing buffer (1 mg bovine serum albumin (BSA)/ml phosphate buffered saline solution (PBS), pH 7.4), incubated with rat anti-mouse-antibody Ep-CAM (BD Biosciences Pharmigen, diluted 1:1,000 in 10 mg BSA/ml PBS) overnight in a moisture chamber at 4° C. and in the dark at room temperature for one hour with goat anti-rat Cy-3 conjugated antibody (Chemicon, diluted 1:200 in 10 mg BSA/ml PBS) following washing steps with buffer. After subsequent washing, the sections were inserted into water-soluble medium (Glycergel, DAKO) containing 4′,6-diamidino-2-phenylindol (DAPI, Serva) for core dyeing and in Bicyclo(2,2,2,)-1,4-diazaoctane (DABCO, Sigma) for dye removal. Locations with the Ep-CAM were made visible with a UV fluorescence microscope and documented with a digital camera. Unspecific dyeings were confirmed by leaving out the primary antibody.

Table 6: Increased genes with relation to the ECM change in non-small-celled lung carcinomas. Fold Change (P<0.1) was calculated by means of Affymetrix software (DMT 3.0). * Genes only determined as “present” in lung adenocarcinomas.

Table 7: Validation of microarray data by means of qRT-PCR. FC (fold change) determined by qRT-PCR. Semi-quantification of the bands was done with the Kodak 1 D 3.5 network software.

FIG. 4: Expression of key genes associated with proteolytic activity in lung cancer. A. Increased Bsg, MMP12 and ADAM19 in tumoral lung tissue. Controls are portrayed by striped squares. B. Induction of basigin, on ADAM 19 and of MMP12 in lung adenocarcinoma. Immunoblots show increased Bsg, MMP12 and ADAM19 in lung tumours. L. Protein conductor. 100 μg of the protein extracts of lung controls and of lung tumours were used for each track.

FIG. 5: Expression of Ep-Cam in lung adenocarcinoma. A. Dapi dyeing of nuclei and Ep-Cam dyeing of lung adenocarcinoma. Membrane dyeing is observed in the respiratory epithelium, edging the terminal bronchiae and bronchiolae. B. Ep-Cam dyeing marks the tumour foci. The epithelium of pseudo-papillary or papillary formations shows a strongly positive dyeing. Response is homogeneous and membrane-bound. The hyperplastic alveolar epithelium in the foci also dyes positively.

FIG. 6: Validation of microarray data by means of qRT-PCR. FC (fold change) determined by qRT-PCR. Semi-quantification of the bands was done with the Kodak 1D 3.5 network software.

FIG. 4a: Expression of key genes associated with proteolytic activity in lung cancer.

FIG. 4b: Induction of basigin, ADAM19 and MMP12 in the lung adenocarcinoma

FIG. 5: Expression of Ep-Cam in the lung adenocarcinoma

FIG. 6: Validation of microarray data by means of qRT-PCR.

TABLE 6

| | | | Fold change | | |
|---|---|---|---|---|---|
| | | | (tumor stage/months-old) | | |
| Affymetrix | | | | Intermediate/ | |
| probe set ID | Gene symbol | Gene Description | Early/4- | 7- | Late/11- |
| 103554_at | ADAM19 | a Disintegrin and metalloproteinase 19 | NC | 2.6 | 2.2 |
| 95338_s_at | Mmp12 | Metalloproteinase 12 | NC | 2.1 | 2.4 |
| 101881_g_at | Col18a1 | Procollagen, type XVIII alpha1 | 2.21 | 2.3 | 2.5 |
| 99637_at | Col15a1 | Procollagen, type XV alpha1 | −1.5 | −1.5 | 5.0 |
| 103005_s_at | CD44 | CD 44 | 3.1 | 3.7 | 4.0 |
| 101078_at | Bsg | Basigin | 1.9 | 2.2 | 1.8 |
| 102353_at | Itgb2 | Integrin beta 2 | 2.2 | 2.6 | 4.0 |
| 104308_at | Itgax | Integrin alpha 10 | 1.4 | 2.0 | 3.3 |
| 100428_at | Lamc2 | Laminin gamma 2 | 1.6 | 3.5 | 4.2 |
| 92759_at | Lamb3 | Laminin beta 3 | 1.6 | 1.6 | 3.3 |
| 93934_at | Cldn2 | Claudin 2* | NC | 6.5 | 20.3 |
| 94493_at | Cldn3 | Claudin 3 | 2.0 | 3.5 | 2.4 |
| 99561_f_at | Cldn7 | Claudin 7 | 1.5 | 2.4 | 2.1 |
| 94270_at | Krt1-18 | Keratin complex 1, acidic gene 18 | NC | 3.4 | 2.7 |
| 101009_at | Krt2-8 | Keratin complex 2, basic gene 8 | NC | 2.2 | 1.7 |
| 99582_at | tacstd1 | tumor associated calcium signal transducer 1 | NC | 2.4 | 1.8 |
| 160651_at | tacstd2 | tumor associated calcium signal transducer 2 | NC | NC | 2.4 |
| 95453_f_at | S100 a1 | S100 calcium binding protein A1 | 1.8 | 2.1 | 2.0 |
| 98600_at | S100 a11 | S100 calcium binding protein A11 | 2.2 | 2.5 | 1.8 |

TABLE 7

| Genes | RefSeq | Fold change (FC) |
|---|---|---|
| ADAM19 | NM 009616 | +2.65□ 1.01 |
| Bgn | NM_009768 | +2.84□ 1.78 |
| CD44 | NM_009851 | +2.61□ 1.23 |
| ItgaX | NM_021334 | +2.82□ 0.54 |
| Lamc2 | NM_008485 | +4.61□ 1.53 |
| B-Actin | NM_008084 | −1.03 □ 0.75 |

1. Means for the identifying, marking and treating tumour cells, wherein a biological or biotechnological system is put into contact with at least one substance, which has been at least partly dissolved and has affinity to at least one of genes ADAM19, Mmp12, Col18a1, Col15a1, CD44, Bsg, Itgb2, Itgax, Lamc2, Lamb3, Alcam, Cidn2, Cidn3, Cidn7, Krt1-18, Krt2-8, tacstd1, tacstd2, S100a1, S100a11 and/or their variants and/or parts thereof and/or their mRNA and/or their gene products and/or cleavage products, polypeptides or peptides derived therefrom and wherein the substance is connected with a marker.

2. Method according to claim 1, wherein the biological or biotechnological system has been formed as a organism, cell tissue, cell, integral part of a cell, DNA, RNA, cDNA, mRNA, cRNA, protein and/or peptide or structures derived from them or contains the latter.

3. Method according to one of the aforementioned claims, wherein the biological or biotechnological system entails lung tumour cells and/or oligonucleotide libraries.

4. Method according to one of the aforementioned claims, wherein at least one substance has been formed as an oligonucleotide, protein, peptide or structure derived therefrom.

5. Method according to one of the aforementioned claims, wherein at least one substance has been formed as a monoclonal antibody and/or polyclonal antibody and/or antibody fragment.

6. Method according to one of the aforementioned claims, wherein at least one substance has been formed as a humanised and/or bi-specific antibody or as a humanised and/or bi-specific antibody fragment.

7. Method according to one of the aforementioned claims, wherein the marker has been formed as an element, molecule and/or ion or has been composed thereof.

8. Method according to one of the aforementioned claims, wherein the marker has been formed as a dye, contrast agent, chemo-therapeutic, radionuclide, toxin, lipid, carbohydrate, biotin, peptide, protein, microparticle, vesicle, polymer, hydro-gel, cell organelle, virus and/or whole cell or entails the latter.

9. Method according to one of the aforementioned claims, wherein the marker has been formed as a marked secondary antibody or Protein A or Protein G or structured derived therefrom.

10. Method according to one of the aforementioned claims, wherein the marker entails hyaluronan or structures derived therefrom.

11. Method according to one of the aforementioned claims, wherein the marker has connected chemically, electrostatically and/or via hydrophobic interactions with at least one substance.

12. Method according to one of the aforementioned claims, wherein the connection between at least one substance and the marker is co-valent.

13. Method according to one of the aforementioned claims, wherein a plurality of substances with affinity to the genes Bsg, Cidn2, Tnfsf9, ADAM-19 or their mRNA sequences or their gene products are used.

14. Method according to one of the aforementioned claims, wherein the affinity has been designed via an association constant Ka>1,000 M−1.

15. Method according to one of the aforementioned claims, wherein it contains a PCR, in vitro transcription, RT-PCR, gel electrophoresis, Western Blot, Northern Blot, Southern Blot, ELISA, FACS measurement, chromatographic separation, UV microscopy, immunohistochemistry, screening of solid-phase bound molecules, cells or tissues, statistical evaluation and/or biosensory examination.

16. Method according to claim 14, wherein the solid-phase bound molecules, cells or tissues have been immobilised on a planar surface.

17. Method according to claims 14-15, wherein the solid-phase bound molecules, cells or tissues have been immobilised with local addressing.

18. Method according to claims 14-15, wherein the solid-phase bound molecules, cells or tissues have been formed as at least one substance with affinity to at least one of the genes ADAM19, Mmp12, Col18a1, Col15a1, CD44, Bsg, Itgb2, Itgax, Lamc2, Lamb3, Alcam, Cidn2, Cidn3, Cidn7, Krt1-18, Krt2-8, tacstd1, tacstd2, S100a1, S100a11 and/or their variants and/or parts thereof and/or their mRNA and/or their gene products and/or cleavage products, polypeptides or peptides derived therefrom.

19. Method according to claims 15-18, wherein the planar surface has been formed as a glass, plastic or membrane surface.

20. Method according to claim 19, wherein the membrane surface has essentially formed of cellulose, nitro-cellulose or PVDF.

21. Method according to claims 15-20, wherein the locally addressed, bound oligonucleotides have been formed as a DNA library.

22. Method according to claims 15-21, wherein the surface has been put into contact with a solution with a biological or biotechnological system according to claims 2-3 and the components associated out of the solution have been made visible on the surface.

23. Method according to claim 22, wherein dyeing reagents, marked oligonucleotides, proteins and/or peptides are used for making visible.

24. Method according to claims 22-23, wherein the proteins have been formed as antibodies, enzymes, streptavidin and/or as parts thereof.

25. Method according to one of the aforementioned claims, wherein signals emanating from the marker are read out with the help of an optically or scintigraphically sensitive apparatus.

26. Means for identifying, marking and treating tumour cells pursuant to a method according to one of the aforementioned claims, wherein it contains at least one of the substances with an affinity pursuant to claim 1 and, if applicable claim 14, and pursuant to the features according to one of the claims 4-8 and at least one of the substances is connected with a marker pursuant to one of the claims 7-9, if applicable via a connection pursuant to claims 11-12, and the marker substances is at least partly soluble.

27. Means pursuant to claim 26, wherein it is soluble in aqueous solutions.

28. Test kit for identifying, marking and treating epithelial lung tumour cells pursuant to a method according to claims 1-25, wherein it contains at least one means pursuant to one of the claims 26-27.

29. Test kit according to claim 28, wherein it contains a bearing agent on which at least one substance with affinity to at least one of the genes ADAM19, Mmp12, Col18a1, Col15a1, CD44, Bsg, Itgb2, Itgax, Lamc2, Lamb3, Alcam, Cidn2, Cidn3, Cidn7, Krt1-18, Krt2-8, tacstd1, tacstd2, S100a, S100a11 and/or their variants and/or parts thereof and/or their mRNA and/or their gene products and/or cleavage products, polypeptides or peptides derived therefrom has been immobilised.

30. Test kit according to claim 29, wherein it entails
a) a carrier on which at least one substance with affinity to ADAM19 and/or its variants and/or parts thereof and/or of its mRNA and/or its gene products and/or cleavage products, polypeptides or peptides derived therefrom has been immobilised and
b) a primer pair with the sequences 5′-cgatggcggctgcatcatggc-3′ and 5′-ccaccagcttgcactggtggc-3′ or structures derived therefrom.

31. Test kit according to claim 29, wherein it entails
a) a carrier on which at least one substance with affinity to TIMP-3 and/or its variants and/or parts thereof and/or of its mRNA and/or its gene products and/or cleavage products, polypeptides or peptides derived therefrom has been immobilised and
b) a primer pair with the sequences 5′-gatgccccacgtgcagtacat-3′ and 5′-tgctgatgctcttgtctgggg-3′ or structures derived therefrom.

32. Test kit according to claim 29, wherein it entails
a) a carrier on which at least one substance with affinity to col17a1 and/or its variants and/or parts thereof and/or of its mRNA and/or its gene products and/or cleavage products, polypeptides or peptides derived therefrom has been immobilised and
b) a primer pair with the sequences 5′-ggctgagctggacggctacag-3′ and 5′-gggttcaccacgaggtcccat-3′ or structures derived therefrom.

33. Test kit according to claim 29, wherein it entails
a) a carrier on which at least one substance with affinity to Cav1 and/or its variants and/or parts thereof and/or of its mRNA and/or its gene products and/or cleavage products, polypeptides or peptides derived therefrom has been immobilised and
b) a primer pair with the sequences 5′-gcatcaagagcttcctgattg-3′ and 5′-ccagactgtcaaacatagatg-3′ or structures derived therefrom.

34. Test kit according to claim 29, wherein it entails
a) a carrier on which at least one substance with affinity to Bsg and/or its variants and/or parts thereof and/or of its mRNA and/or its gene products and/or cleavage products, polypeptides or peptides derived therefrom has been immobilised and
b) a primer pair with the sequences 5′-aaccgggcaccatccaaacct-3′ and 5′-attgcctcttcttccccagtg-3′ or structures derived therefrom.

35. Test kit according to claim 29, wherein it entails
a) a carrier on which at least one substance with affinity to CD44 and/or its variants and/or parts thereof and/or of its mRNA and/or its gene products and/or cleavage products, polypeptides or peptides derived therefrom has been immobilised and
b) a primer pair with the sequences 5′-cggctccaccatcgagaagag-3′ and 5′-gttgtgggctcctgagtctga-3′ or structures derived therefrom.

36. Test kit according to claim 29, wherein it entails
a) a carrier on which at least one substance with affinity to ItgaX and/or its variants and/or parts thereof and/or of its mRNA and/or its gene products and/or cleavage products, polypeptides or peptides derived therefrom has been immobilised and
b) a primer pair with the sequences 5′-ccctcaaatatgagacccacc-3′ and 5′-ggattcctgggtaaagatgac-3′ or structures derived therefrom.

37. Test kit according to claim 29, wherein it entails
a) a carrier on which at least one substance with affinity to Lamc2 and/or its variants and/or parts thereof and/or of its mRNA and/or its gene products and/or cleavage products, polypeptides or peptides derived therefrom has been immobilised and
b) a primer pair with the sequences 5′-gctggaaggcaggatcgagca-3′ and 5′-ttcctgccagactcaggcgca-3′ or structures derived therefrom.

38. Test kit according to claim 29, wherein it entails
a) a carrier on which at least one substance with affinity to TM4sf2 and/or its variants and/or parts thereof and/or of its mRNA and/or its gene products and/or cleavage products, polypeptides or peptides derived therefrom has been immobilised and
b) a primer pair with the sequences 5′-gttgattggcatgctgctggc-3′ and 5′-gcagggatagtatgtactgtg-3′ or structures derived therefrom.

39. Test kit according to claim 29, wherein it entails
a) a carrier on which at least one substance with affinity to CAP-1 and/or its variants and/or parts thereof and/or of its mRNA and/or its gene products and/or cleavage products, polypeptides or peptides derived therefrom has been immobilised and
b) a primer pair with the sequences 5′-cacgctcagcactgtttgcac-3′ and 5′-cacttgtagatgtaagccacc-3′ or structures derived therefrom.

40. Test kit according to claim 29, wherein it entails
a) a carrier on which at least one substance with affinity to Vtn and/or its variants and/or parts thereof and/or of its mRNA and/or its gene products and/or cleavage products, polypeptides or peptides derived therefrom has been immobilised and
b) a primer pair with the sequences 5′-aagtggagcaacaggaggaga-3′ and 5′-caacattgtctggtatgccac-3′ or structures derived therefrom.

41. Test kit according to claim 29, wherein it entails
a) a carrier on which at least one substance with affinity to Ccr5 and/or its variants and/or parts thereof and/or of its mRNA and/or its gene products and/or cleavage products, polypeptides or peptides derived therefrom has been immobilised and
b) a primer pair with the sequences 5′-gtcagaacggtcaactttggg-3′ and 5′-gttgtagggagtccagaagag-3′ or structures derived therefrom.
